(19) United States
(12) Patent Application Publication
(10) Pub. No.: US 2025/0099570 A1
(43) Pub. Date: Mar. 27, 2025

(54) ANTIBODIES TARGETING MARBURGVIRUS GLYCOPROTEINS AND USES THEREOF

(71) Applicants: University of Maryland, College Park, College Park, MD (US); Government of the United States of America, as represented by the Secretary of Commerce, Gaithersburg, MD (US)

(72) Inventors: Gilad Adi Ofek, Rockville, MD (US); Benjamin M. Janus, Columbia, MD (US); Thomas R. Fuerst, Germantown, MD (US); Thomas E. Cleveland, IV, Monrovia, MD (US)

(21) Appl. No.: 18/892,905

(22) Filed: Sep. 23, 2024

Related U.S. Application Data
(60) Provisional application No. 63/584,677, filed on Sep. 22, 2023.
(60) Provisional application No. 63/648,954, filed on May 17, 2024.
(60) Provisional application No. 63/650,748, filed on May 22, 2024.

(51) Int. Cl. A61K 39/12; A61P 31/14; C07K 14/005; C07K 16/10; C12N 7/00; G01N 33/569; G01N 33/68

(52) U.S. Cl. CPC A61K 39/12; A61P 31/14; C07K 14/005; C07K 16/10; C12N 7/00; G01N 33/56983; G01N 33/6854; C07K 2317/565; C07K 2317/94; C12N 2760/14034; G01N 2333/08

(57) ABSTRACT

The present disclosure relates generally to antibodies, such as antibodies and fragments thereof, that bind to the marburgvirus glycoprotein. Such antibodies have many applications including their use as antiviral drugs for treatment and prevention of diseases resulting from marburgvirus infection.

### TECHNICAL FIELD

The present disclosure generally relates to antigen binding proteins, for example, antibodies and fragments thereof, that bind to the marburgvirus glycoprotein (GP) antigen. Such proteins, and fragments thereof, have many applications including their use as antiviral drugs for treatment and prevention of diseases resulting from marburgvirus infection.

### BACKGROUND

Marburgviruses were the first filoviruses characterized to emerge in humans in 1967 and have led to multiple outbreaks since then with average case fatality rates of ˜50%. Although a vaccine and monoclonal antibody countermeasures have been approved for clinical use against the related Ebola virus, these are ineffective against marburgviruses or other filoviruses. Thus, the development of novel therapeutics targeting marburgviruses, or any additional viruses in general, are urgently needed.

### SUMMARY

The present disclosure relates to antigen binding proteins, e.g. antibodies, and fragments thereof, that bind to the marburgvirus glycoprotein, and their use in treatment and/or prevention of viral infections. While the disclosure below relates to antigen (GP) binding proteins in general, the disclosure is described below for marburgvirus GP reactive antibodies and fragments thereof.

It should be noted, that the marburgvirus genus contains viruses belonging to two main isolates/strains: (i) Marburg (commonly abbreviated “MARV”) and (ii) Ravn, commonly abbreviated “RAVV”). As used herein, “MARV-antibody” or “MARV-antibodies” refer to all classes of marburgvirus GP reactive antibodies, including those that are cross-reactive to multiple isolates.

Additionally, while the disclosure below is directed to marburgvirus antibodies (MARV-antibodies) that bind to the viral glycoprotein (GP), it is understood that said disclosure can be applied equally as well to other filoviruses including, but not limited to, those having a corresponding GP.

In an embodiment, MARV-antibodies, or fragments thereof, are provided that bind to the viral GP. Such antibodies include, for example, polyclonal, monoclonal or humanized antibodies. In another embodiment the antibody, or fragment thereof, is a monkey-human chimeric antibody. MARV-antibodies also include Fab fragments, single chain antibodies, bivalent antibodies and multivalent antibodies that bind to the Marburgvirus GP. Such antibodies, or fragments thereof, may have the ability to neutralize the viral activity within an infected subject. The term “neutralize” or “neutralizing antibody” refers to an antigen binding protein or antibody that binds to an antigen, i.e, viral GP antigen, and prevents or reduces entry of the virus into the host cell and thus reduces the biological effect of the marburgvirus or by preventing or reducing the release of the budding marburgvirus. This can be done, for example, by directly blocking a binding site on the virus or by binding to virus and altering the viruses' ability to bind through indirect means.

Additionally, the binding of neutralizing antibodies, or fragments thereof, to the viral GP may have the ability to protect the host from viral infection by recruiting other immune effector functions to sites of infection. Additionally, the binding of non-neutralizing antibodies, or fragments thereof, to the viral GP may have the ability to protect the host from viral infection by recruiting other immune effector functions to sites of infection, or by other means. Additionally, the binding of different combinations of antibodies, neutralizing and/or non-neutralizing, or fragments thereof, to the viral GP may have the ability to protect the host from viral infection by recruiting other immune effector functions to sites of infection, or by other means. Such combinations may in some cases yield cooperative or synergistic improvements in antibody efficacy as well as reduced likelihood of viral resistance.

Another aspect of the present disclosure provides an anti-viral composition comprising a MARV-antibody, or fragment thereof, as disclosed herein, as an active ingredient. As used herein, the term “anti-viral composition” refers to a composition able to prevent infection or re-infection with a viral pathogen. In a non-limiting embodiment, the virus is marburgvirus, and the antiviral composition is able to reduce the severity of symptoms or eliminate the symptoms, associated with infection with a marburgvirus or substantially or completely remove the disease caused by infection with marburgvirus. Thus, the anti-viral compositions disclosed herein may be administered prophylactically to a subject, e.g., a human, before infection with marburgvirus, or may be therapeutically administered to subjects after infection with marburgvirus.

The anti-viral compositions provided herein may be prepared in any suitable and pharmaceutically acceptable formulation. It may be provided in the form of an immediately administrable solution or suspension, or a concentrated crude solution suitable for dilution before administration or may be provided in a form capable of being reconstituted, such as a lyophilized, freeze-dried, or frozen formulation.

The anti-viral composition may contain a pharmaceutically acceptable carrier in order to be formulated. The carrier typically includes a diluent, an excipient, a stabilizer, a preservative, and the like. In a specific embodiment of the invention, the anti-viral composition is formulated for intranasal administration. In another embodiment, the anti-viral composition is formulated for systemic administration.

Another aspect pertains to compositions comprising nanoparticles and the disclosed MARV-antibodies or fragments thereof. Nanoparticles can be created from biological molecules or from non-biological molecules. In some cases, the MARV-antibodies are crosslinked to a polymer or lipids on the nanoparticle surface. In embodiments, the MARV-antibodies, or fragments thereof, are adsorbed onto the nanoparticle surface. In some embodiments, the disclosed MARV-antibodies are adsorbed onto the nanoparticle surface and then crosslinked to the nanoparticle surface. In some embodiments, disclosed MARV-antibodies are encapsulated into the nanoparticle. Such nanoparticles, or nanoliposomes may be incorporated into anti-viral compositions. In some instances, the disclosed antibodies, or antibody fragments, may be genetically fused to nanoparticle genes.

A method of treating a subject is provided that includes administering a disclosed anti-viral composition comprising one or more MARV-antibodies, or fragments thereof, as described herein, to a subject in need thereof. In a non-limiting embodiment, the antiviral composition is able to reduce the severity of symptoms or eliminate the symptoms of marburgvirus infection, or substantially or completely remove the disease caused by marburgvirus infection. Thus, for methods of treatment provided herein, the anti-viral compositions may be administered prophylactically to a subject, e.g., a human, before infection with marburgvirus, or may be therapeutically administered to subjects after infection with marburgvirus.

The disclosed anti-viral composition may be administered in a number of ways. For example, the disclosed anti-viral composition can be administered intramuscularly, intranasally, orally, intravenously, subcutaneously, transdermally (e.g., by microneedle), intraperitoneally, ophthalmically, sublingually, or by inhalation. In a specific embodiment, the anti-viral is administered intranasally.

In still yet another aspect, a diagnostic composition that employs the use of a MARV-antibody and methods for detecting the presence of a virus in a subject sample are provided. In an embodiment, the MARV-antibody detects the presence of viral GP, in a subject sample, and can be used to distinguish marburgvirus-infected and uninfected subjects from each other by bringing the same into contact with a sample and measuring the extent of reaction there between.

The present disclosure provides a kit that includes the MARV-antibody compositions, as described herein. Such kits may be used for diagnostic, or treatment uses. In one specific aspect the kit further includes instructions for the diagnosis, treatment and/or prophylaxis of marburgvirus infection. The anti-viral compositions may, if desired, be presented in a pack or dispenser device which may contain one or more-unit dosage forms containing the aptamer antiviral composition. In a specific embodiment, the dispenser may be one to be used for intranasal administration of the anti-viral composition. In a specific embodiment, the dispenser may be one to be used for intramuscular administration of the anti-viral composition. The pack may for example include metal or plastic foil, such as a blister pack. The pack or dispenser device may be accompanied by instructions for administration to subjects, especially humans, or for diagnostic purposes.

### BRIEF DESCRIPTION OF THE DRAWINGS

FIG. 1A-C. Multivalent prime-boost immunization of Rhesus macaques. (FIG. 1A) Immunization regimens for three Rhesus macaques, each receiving four immunizations at 2-week intervals. Serum bleeds were taken at day 0 (preimmune) and 7 days after each immunization. (FIG. 1B) Schematics of immunogens used in the study, including virus-like particles (VLPs) composed of GP, VP40, and NP, recombinant full-length GP ectodomains (GPΔTM), and recombinant GP ectodomains lacking mucin-like domains (GPΔMuc). (FIG. 1C) Intra-genus sequence identity matrix for full-length GPs corresponding to study immunogens.

FIG. 2A-D. Autologous and heterologous serum antibody binding and neutralization titers. Study day 49 serum ELISA binding profiles for each animal to autologous (FIG. 2A) or heterologous (FIG. 2B) recombinant GPΔMuc proteins. Shown are means of technical duplicates with error bars indicating standard deviation. Study day 49 serum neutralization profiles for each animal against autologous GP pseudotyped MLV viruses (FIG. 2C) or heterologous GP pseudotyped MLV or VSVΔG viruses (FIG. 2D). Shown are means of technical duplicates with error bars indicating standard deviation. Results are of representative experiments repeated two or more times for orthomarburgvirus targets and 1-2 times for orthoebolavirus targets.

FIG. 3A-C. Antigen-specific memory B-cell sorting with a heterologous orthomarburgvirus GP probe. (FIG. 3A) Sequence divergence of the heterologous RAVV GPΔMuc B-cell sorting probe from autologous MARV Musoke and Angola immunogens. (FIG. 3B) Heterologous RAVV GPΔMuc probe competition for NHP1 day 49 serum neutralizing antibodies, against MARV-MLV pseudoviruses. Pre, preimmune. Shown are single replicates of a representative experiment repeated two times. (FIG. 3C) Memory B-cell sorting pipeline for RAVV GPΔMuc double-positive B cells with the phenotype CD27+, IgG+, CD20+, Aqua Dead−, CD3−, CD8−, CD14−, and IgM−.

FIG. 4A-C. Antibody heavy and light chain sequence features. (FIG. 4A) Heavy and light chain gene usage of the 33 expressed monoclonal antibodies. (FIG. 4B) Heavy chain and light chain somatic mutation frequencies are shown as percentages of total amino acids in variable regions. (FIG. 4C) Heavy chain CDR3 length distribution across the antibody panel.

FIG. 5A-C. Antibody binding and pseudovirus neutralization. (FIG. 5A) Antibody ELISA binding EC50s to RAVV GPΔMuc and neutralization IC50s against MLV-MARV Musoke pseudoviruses calculated from individual plots shown in Fig. S4 and S5. Antibody CM1, CM2, CM11, and CM12 lineage variants are shaded light orange, orange, teal, and cyan, respectively. (FIG. 5B) ELISA binding profiles of antibody CM1 and CM2 lineage variants to recombinant RAVV GPΔMuc. Shown are means of technical duplicates with error bars indicating standard deviation. Results are of representative experiments repeated at least three times. (FIG. 5C) Neutralization of Musoke MLV-MARV pseudoviruses by antibody CM1 and CM2 lineage variants. Shown are means of technical duplicates with error bars indicating standard deviation. Results are of representative experiments repeated at least three times.

FIG. 6A-E. Epitope mapping by overlapping pepscan analysis. (FIG. 6A) SDS-PAGE Western blots of RAVV GPΔMuc probed with five mAbs observed to give detectable recognition of denatured GP. (FIG. 6B) Schematic of overlapping 15-mer peptides across the GP1 and GP2 subunits of RAVV GPΔMuc that were used for pepscan analyses. (FIG. 6C) Overlapping peptide ELISA binding competition for RAVV GPΔMuc recognition by mAbs CM10, CM11.1, CM12.1, and CM13, focused on regions of GP1 and GP2 that exhibited competition. Shown are single replicates of representative experiments performed 1-2 times. (FIG. 6D) Direct ELISA binding of mAbs CM10, CM11.1, CM12.1, and CM13 to overlapping peptides, focused on regions defined in C. Shown are single replicates of representative experiments performed two or more times. (FIG. 6E) Sequence alignments of the GP1 epitope of mAb CM13 across filoviruses, top, and of the GP2 epitope of mAbs CM10, CM11, and CM12 across orthomarburgviruses, bottom. Overlapping epitopes of previously characterized mAbs m21D10, 30G4, and MR228 are shown as bars above.

FIG. 7A-E. Antibody binding competition groups. (FIG. 7A) Shown are %-binding values of each antigenic benchmark mAb to biotinylated RAVV GPΔMuc in the presence of each competitor mAb. Percentages are calculated relative to the capture of biotinylated RAVV GPΔMuc in the absence of a competing mAb. Orthoebolavirus-specific mAb CA45 was used as a negative control. Results are of representative experiments performed 1-2 times with 1-2 replicates. (FIG. 7B) Plots of the %-binding competition against each benchmark mAb. (FIG. 7C) NSEM 2D class averages of CM1.1 Fab in complex with recombinant RAVV GPΔMuc protein. (FIG. 7D) NSEM 2D class average of CM2.1 Fab in complex with recombinant RAVV GPΔMuc protein. (FIG. 7E) Antigenic footprints of benchmark mAbs MR191, CM1.1, CM10, and CM13, mapped onto the surface of RAVV GPΔMuc (PDB ID 6BP2). GP1 and GP2 are colored blue and orange, respectively. The predicted RBR is colored green, based on the epitope of MR191. The first ordered residues within the N terminus of GP2 (GP2-N) are colored red and the CM13 epitope is colored yellow.

FIG. 8. Cross-filovirus GP recognition. ELISA binding profiles of antibodies CM16 and CM20 to RAVV, EBOV, SUDV, and BDBV GPΔMuc proteins. Orthomarburgvirus-specific mAb MR191 and orthoebolavirus-specific cross-reactive mAb CA45 were analyzed as controls. Shown are means of technical duplicates with error bars indicating standard deviation. Results are of representative experiments repeated at least three times.

FIG. 9A-C. Serum antibody recognition of autologous GPs. ELISA binding profiles of serum bleeds from each animal taken on study days 0 (preimmune), 21, 35, and 49 to recombinant MARV GPΔMuc (FIG. 9A), SUDV GPΔMuc (FIG. 9B), and EBOV GPΔMuc (FIG. 9C). Shown are means of technical duplicates with error bars indicating standard deviation. Results are of representative experiments repeated two or more times

FIG. 10A-C. Serum antibody binding ED50s to autologous GPs across study days 21, 35, and 49. Progression of serum ELISA 50% binding effective dilutions (ED50s) across study days 21, 35, and 49 to recombinant MARV GPΔMuc (FIG. 10A), SUDV GPΔMuc (FIG. 10B), and EBOV GPΔMuc (FIG. 10C). Binding ED50, log ED50, and log SE were calculated in Graphpad Prism using the plots shown in Fig. S1. Error bars represent log SE (log Standard Error).

FIG. 11. NHP1, NHP2, and NHP3 day 49 serum neutralization of heterologous marburgvirus RAVV GP MLV psuedoviruses. Shown are means of technical duplicates with error bars indicating standard deviation. Results are of a representative experiment repeated three times.

FIG. 12. Antibody ELISA binding profiles to RAVV GPΔMuc. ELISA binding profiles of each individual antibody to RAVV GPΔMuc. Shown are means of technical duplicates with error bars indicating standard deviation. Results are of representative experiments performed at least two times for antibodies that exhibited detectable binding.

FIG. 13. Antibody neutralization of Musoke MARV-MLV pseudoviruses. Neutralization profiles of each individual antibody against MARV-GP MLV pseudoviruses. Shown are means of technical duplicates with error bars indicating standard deviation. Results are of representative experiments performed at least two times for antibodies that exhibited detectable neutralization.

FIG. 14. Cartoon representation of the crystal structure of RAVV GPΔMuc in complex with RBR-directed neutralizing antibody MR191 (PDB ID 6BP2). GP1 and GP2 are colored blue and orange, respectively, and MR191 is colored gray.

FIG. 15. MARV pseudovirus release in presence of antibodies. MARV-VSV psuedovirus titers released into cell culture supernatants when produced in the presence of listed antibodies, shown as a percentage relative to titers released in the absence of antibodies. Values are means across two independent experiments, with 4-8 technical replicates per antibody. Error bars indicate standard error of the mean. Ebolavirus specific antibody CA45 was used as a negative control.

### DETAILED DESCRIPTION

The present disclosure relates to antigen binding proteins, e.g., antibodies, and fragments thereof, that bind to the marburgvirus (MARV) glycoprotein (GP). Such antibodies have many applications including their use as antiviral drugs for treatment and prevention of diseases resulting from marburgvirus infection.

It should be noted, that the marburgvirus genus contains viruses belonging to two main isolates/strains: (i) Marburg (commonly abbreviated “MARV”) and (ii) Ravn, commonly abbreviated “RAVV”). As used herein, “MARV-antibody” or “MARV-antibodies” refer to all classes of marburgvirus GP reactive antibodies, including those that are cross-reactive to multiple marburgvirus isolates.

While the disclosure below is directed to MARV-antibodies that bind to the marburgvirus GP protein, it is understood that said disclosure can be applied equally as well to other viruses having corresponding GP proteins including, but not limited to, filoviruses of the genus Ebolaviruses and Marburgviruses, including the ebolaviruses Zaire (EBOV), Sudan (SUDV), and Bundibugyo (BDBV), and the marburgviruses Marburg (MARV) and Ravn (RAVV).

In an embodiment, MARV-antibodies, or fragments thereof, include, but are not limited to those that bind to the marburgvirus viral GP antigen, thereby blocking interaction between the viral receptor binding region and the cell receptor. In an embodiment, the MARV-antibodies, or fragments thereof, include polyclonal, monoclonal, or humanized antibodies including humanized monoclonal antibodies. In another embodiment the antibodies, or fragments thereof, include monkey-human chimeric antibodies. MARV-antibodies also include Fab fragments, single chain antibodies, bivalent antibodies, and multivalent antibodies. In a specific embodiment, the disclosed MARV-antibodies, which were derived from immunized Rhesus macaque B-cells, may be humanized using techniques well known to those of skill in the art. As used herein “fragments thereof” include, for example, fragments of the disclosed marburgvirus antibodies that retain their ability to bind to the viral GP antigen.

In certain embodiments, the MARV-antibodies comprise an immunoglobulin molecule of at least one of the IgG1, IgG2, IgG3, IgG4, Ig E, IgA, IgD, and IgM isotype. In certain embodiments, antibodies comprise a human kappa light chain and/or a human heavy chain. In certain embodiments, the heavy chain is of the IgG1, IgG2, IgG3, IgG4, IgE, IgA, IgD, or IgM isotype. In an embodiment, said immunoglobulins comprise one or more of the heavy and/or light chain CDR regions identified for monoclonal antibodies CM1.1, CM1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28, as well as amino acid sequences that have at least have at least 50%, 60%, 70%, 80%, 85%, 90%, 95%, 96%, 97%, 97%, 99%, or above 99% identity to the disclosed CDR amino acid sequences.

In a specific embodiment, the MARV-antibodies include those comprising the heavy chain amino acid sequences of the disclosed monoclonal antibodies CM1.1, CM1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28 as well as amino acid sequences that have at least 50%, 60%, 70%, 80%, 85%, 90%, 95%, 96%, 97%, 97%, 99%, or above 99% identity to the disclosed heavy chain amino acid sequences.

In another embodiment, the MARV-antibodies include those comprising the light chain amino acid sequences of the disclosed monoclonal antibodies CM1.1, CM1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28 as well as amino acid sequences that have at least 50%, 60%, 70%, 80%, 85%, 90%, 95%, 96%, 97%, 97%, 99%, or above 99% identity to the disclosed light chain amino acid sequences.

Antibody variable regions typically comprise the same general structure of relatively conserved framework regions (FR) joined by three hyper-variable regions, referred to as complementarity determining regions or CDRs. The CDRs from the two chains of each pair typically are aligned by the framework regions, which enable binding to a specific epitope. In a specific embodiment, MARV-antibodies also include those comprising one or more of the heavy and/or light chain CDR regions identified for monoclonal antibodies CM1.1, CM1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28, as well as amino acid sequences that have at least have at least 50%, 60%, 70%, 80%, 85%, 90%, 95%, 96%, 97%, 97%, 99%, or above 99% identity to the disclosed CDR amino acid sequences.

In an embodiment, the present disclosure relates to any marburgvirus GP antigen binding protein that retains the CDR sequences disclosed herein sufficient to direct binding to the marburgvirus GP antigen. Such antigen binding proteins include, for example, fusion proteins that may include (fuse) a domain (protein or chemical) having novel functional characteristics to the antigen binding domains. In such fusion proteins the domain may be attached to the antigen binding domain covalently or non-covalently. Also included, are nucleic acids encoding such fusion proteins.

The term “identity” or “sequence identity” is known in the art and refers to a relationship between two or more polynucleotide or amino acid sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073 (1988), the teachings of which are incorporated herein by reference. Methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12 (1): 387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference).

The disclosed MARV-antibodies may further include one or more modifications that, for example, increase the stability of the antibody. For example, the antibodies may be pegylated to increase their stability. The MARV-antibodies may also be conjugated to a therapeutic compound thereby providing a delivery device for targeting of the therapeutic compound to the antibody target. In such an instant, MARV-antibodies, identified for their ability to bind to a viral target protein, i.e., viral GP antigen, is conjugated to a drug useful for treatment or prevention of diseases resulting from infection with said Marburgvirus pathogen. The MARV-antibodies may also be modified to include a “label”, e.g., a detectable marker. Such labels include, but are not limited to, radioisotopes or radionuclides, fluorescent labels, enzymatic labels and chemiluminescent labels. In certain embodiments, labels are attached by spacer arms of various lengths to reduce potential steric hindrance.

In still another aspect, a method is provided for preparation of the MARV-antibodies provided herein. In certain embodiments, nucleic acid sequences encoding the antibodies are cloned for expression in mammalian cells. The preparation methods according to the present disclosure may be performed through recombinant DNA technology known in the art. Such methods include, for example, the cloning of nucleic acid sequences encoding for MARV-antibodies, or fragments thereof, into recombinant expression vectors resulting in recombinant expression of the antibodies, or fragments thereof.

In a specific embodiment, the nucleic acid sequences encoding for MARV-antibodies, or fragments thereof, include those comprising the nucleic acids encoding the heavy chain amino acid sequences of the disclosed monoclonal antibodies CM1.1, CM1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28 as well as sequences that have at least 50%, 60%, 70%, 80%, 85%, 90%, 95%, 96%, 97%, 97%, 99%, or above 99% identity to said nucleic acid sequences.

In another embodiment, the nucleic acid sequences encoding for MARV-antibodies, or fragments thereof, include those comprising the nucleic acids encoding light chain amino acid sequences of the disclosed monoclonal antibodies CM1.1, CM1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28 as well as sequences that have at least 50%, 60%, 70%, 80%, 85%, 90%, 95%, 96%, 97%, 97%, 99%, or above 99% identity to said nucleic acid sequences.

In a specific embodiment, the nucleic acid sequences encoding for MARV-antibodies, or fragments thereof, include those comprising nucleic acids encoding one or more of the heavy and/or light chain CDR regions identified for disclosed monoclonal antibodies CM1.1, CM1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28 as well as sequences that each have at least 50%, 60%, 70%, 80%, 85%, 90%, 95%, 96%, 97%, 97%, 99%, or above 99% identity to the said nucleic acid sequences.

In one embodiment, a vector, preferably an expression vector, comprising one or more of the polynucleotides encoding the MARV-antibody of interest, or fragments thereof, is provided. In other embodiments, the vector is introduced into mammalian cells, e.g., CHO cells, to produce the MARV-antibody in supernatant for purification. The resulting MARV-antibody can then be used in pharmaceutical compositions used for treatment or prevention of Marburgvirus-associated diseases.

Methods are well known to one of skill in the art and can be used to construct expression vectors containing the antibody coding sequence with appropriate transcriptional and translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989).

Typically, the vectors are derived from virus, plasmid, prokaryotic or eukaryotic chromosomal elements, or some combination thereof, and may optionally include at least one origin of replication, at least one site for insertion of heterologous nucleic acid, and at least one selectable marker. Embodiments are also contemplated that express MARV-antibodies of interest using artificial chromosomes, e.g., bacterial artificial chromosomes (BACs), yeast artificial chromosomes (YACs), mammalian artificial chromosomes (MACs), and human artificial chromosomes (HACs).

In such vectors, typically, a promoter region would be operably associated with a nucleic acid encoding the MARV-antibody if the promoter was capable of effecting transcription of that nucleic acid. The promoter can be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are known to those skilled in the art.

A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit a-globin, as well as other sequences capable of controlling gene expression in eukaryotic cells. Additional suitable transcription control regions include tissue-specific promoters and enhancers as well as inducible promoters (e.g. promoters inducible tetracyclines).

The term “host cell” means a cell that has been transformed, or is capable of being transformed, with a nucleic acid and thereby expresses a gene of interest. The polynucleotides encoding the MARV-antibodies for therapeutic use may be expressed in any appropriate host cell, preferably a mammalian cell. The host cell can be prokaryotic (bacteria) or eukaryotic (e.g., yeast, insect, plant and animal cells). A host cell strain may be chosen for its ability to carry out desired post-translational modifications of the expressed protein. Such post-translational modifications of the polypeptide include, but are not limited to, acetylation, carboxylation, glycosylation, phosphorylation, hydroxylation, sulfation, lipidation, and acylation.

Exemplary mammalian host cells are COS1 and COS7 cells, NSO cells, Chinese hamster ovary (CHO) cells, NIH 3T3 cells, HEK293 cells, HEPG2 cells, HeLa cells, L cells, MDCK, W138, murine ES cell lines (e.g., from strains 129/SV, C57/BL6, DBA-1, 129/SVJ), K562, Jurkat cells, BW5147 and any other commercially available human cell lines. Other useful mammalian cell lines are well known and readily available from the American Type Culture Collection (ATCC) (Manassas, Va., USA) and the National Institute of General Medical Sciences (NIGMS) Human Genetic Cell Repository at the Coriell Cell Repositories (Camden, N.J., USA).

In a specific embodiment, the MARV-antibody or fragment thereof, encoding nucleic acids may be engineered in expression constructs to be expressed as monoclonal, humanized, chimeric antibodies, Fab fragments, single chain antibodies, bivalent antibodies and multivalent antibodies. In such instances, the nucleic acid sequences, or fragments thereof, disclosed herein may be used to engineer the expression of such antigen (GP) proteins that retain the ability to bind to the GP antigen.

Methods of isolating and purifying MARV-antibodies are also well known in the art, and any known method may be used. Examples thereof may include ultrafiltration, gel filtration, ion exchange chromatography, affinity chromatography, HPLC, hydrophobic chromatography, isoelectric point chromatography, and combinations thereof. In a specific embodiment, the MARV-antibodies, may be engineered to include a tag, such as a HIS-tag, as a means for affinity chromatography.

The present disclosure also provides for a method of identifying MARV-antibodies for use as an anti-viral composition. Such a method comprises immunizing a test animal, for example, a Rhesus macaque, with multivalent prime-boost regimens using antigens from one or more filovirus species. In an embodiment, the antigens are derived from MARV, Sudan (SUDV), and/or ebola (EBOV). Antigen specific B-cells are then sorted for their ability to bind to a filovirus GP.

In a specific embodiment, B-cells are sorted using a heterologous Ravn (RAVV) marburgvirus GP probe. (See, Table III). As disclosed below, the probe in Table III, “RAVN GP dMUC WT (Foldon, TEV, Strep, Linker, 8xHis, Avitag)”, was used to pull out the B cells and associated monoclonal antibodies described herein. The probe was found to function unexpectedly well for sorting B-cells expressing the desired binding properties. This protein design was used for biochemical binding experiments related to RAVN GPΔMuc (also called “RAVV GPΔMuc”). As the sequence in Table III indicates, this probe comprises a designed RAVV GPΔMuc protein that includes a native Ravn GP leader sequence, the Ravn GP ectodomain with a deleted mucin-like domain (ΔMuc), followed by a fibritin foldon trimerization domain, a TEV protease cleavage site, a twin Strep II tag, a linker sequence, an 8xHis tag, and an Avi-tag for biotinylation.

Accordingly, the present disclosure relates to a fusion polypeptide comprising one or more of a fibritin foldon trimerization domain, a TEV protease cleavage site, a twin Strep II tag, a linker sequence, an 8xHis tag, and an Avi-tag an Avi-tag and a marburgvirus or filovirus GP, or a fragment thereof.

In an embodiment the fusion polypeptide is the polypeptide of Table III, a polypeptide having at least 90%-99% homology to the polypeptide of Table III, or fragment thereof (collectively “the polypeptides of Table III”). Said polypeptide, includes variants that retain their ability to function as a probe as described below. The polypeptide of Table III may comprise an appended Avi-tag for use in specific biotinylation of the protein.

Said fusion polypeptides, including the polypeptide of Table III, may be used as a probe for marburgvirus and filovirus GP antigen specific B cell sorting and monoclonal antibody recovery from animals previously infected with a filovirus or immunized with filovirus antigens. In an embodiment the animal is a human. The polypeptides may also be used as a reagent for recovery of serum antibodies specific for marburgvirus and filovirus GP specific antigen from animals, including humans, previously infected with a filovirus or immunized with filovirus antigens. The antibodies may be autologous or heterologous antibodies.

The fusion polypeptides, including the polypeptides of Table III, may be used a reagent for biochemical analysis of binding of antibodies to marburgvirus and filovirus GP; as a reagent for mapping of the epitopes of existing and novel monoclonal antibodies against marburgvirus and filovirus GPs. Said mapping may be performed using competition-based and/or direct mapping assays.

The fusion polypeptides, including those of Table III may be used as a protein reagent, i.e., vaccine antigen, for induction of immune responses against marburgvirus and filovirus GPs and may be further used in analysis and optimization of the RAVV GPΔMuc protein of Table III for use as a vaccine antigen. In an embodiment, the vaccine antigen is a thermostable vaccine antigen. The vaccine formulations of the present disclosure comprise full length and/or a portion of the polypeptide of Table III and a pharmaceutically acceptable carrier or diluent. The pharmaceutically acceptable carrier, diluent or excipient included in the vaccine formulations are well known to those skilled in the art. The vaccine formulations of the present disclosure may also include an adjuvant.

In another aspect, a vaccine kit containing materials useful for the treatment or prevention of marburgvirus and filovirus is provided wherein the vaccine antigen is the polypeptide of Table III. In an embodiment, the kit comprises the necessary components of a vaccine formulation that elicits an immune response to the virus and instructions for its use is also provided herein.

The fusion polypeptides, including those of Table III, may be used for further analysis and optimization of the RAVV GPΔMuc protein, or other filovirus proteins, for improved monoclonal antibody recovery efforts. The polypeptides may also be used for analysis and identification of mutations that facilitate their use as a probe for positive and negative selection of epitope-specific B cells. The fusion polypeptides, including those of Table III, may be used as a diagnostic reagent for detection of marburgvirus and filovirus GP-specific antibodies. The polypeptide may be included in kits for use as a diagnostic reagent for detection of marburgvirus and filovirus GP-specific antibodies

The fusion polypeptides, including those of Table III, may be used for development of hybrid filovirus glycoproteins between species for development of diagnostic or multivalent vaccine antigens. Such hybrid filovirus glycoproteins between species may be used as a B cell probe for monoclonal antibody recovery against species-specific epitopes.

The fusion polypeptides, including those of Table III, may be used for study of the biochemical and biophysical features that facilitate structural analysis alone or in complex with bound antibodies. Such analyses include, for example, cryo-EM, negative-stained EM, and X-ray crystallography.

The fusion polypeptides, including those of Table III, may be used for introduction of alternative glycosylation isoforms. The isoforms can be introduced through expression in genetically engineered Expi293F cells lacking N-acetylglucosaminyltransferase I (GnTI−) activity and therefore lacking complex N-glycans. Alternative glycosylation isoforms may be introduced in glycoengineered CHO cells or through enzymatic processing.

The fusion polypeptides, including those polypeptides of Table III include those engineered to have a C-terminal tags. Such C-terminal tag includes those selected from the group consisting of a foldon trimerization domain, a TEV protease cleavage site, a twin Strep II tag, aHisx8 tag, and a Avi-tag.

Another aspect of the present disclosure provides an anti-viral composition comprising one or more MARV-antibodies, or fragments thereof, as disclosed herein, as an active ingredient. In a non-limiting embodiment, the virus is a marburgvirus, and the antiviral composition is able to reduce the severity of symptoms or eliminate the symptoms of marburgvirus infection, or substantially or completely remove the disease caused by marburgvirus infection. Thus, the anti-viral compositions disclosed herein may be administered prophylactically to a subject, e.g., a human, before infection with marburgvirus, or may be therapeutically administered to subjects after infection with marburgvirus.

Pharmaceutical compositions comprise a therapeutically effective amount of one or more MARV-antibodies dissolved or dispersed in a pharmaceutically acceptable carrier. The preparation of a pharmaceutical composition that contains at least one or more MARV-antibodies and optionally an additional active ingredient will be known to those of skill in the art in light of the present disclosure, as exemplified by Remington's Pharmaceutical Sciences, 18th Ed. Mack Printing Company, 1990. For human administration, it will be understood that preparations should meet sterility, pyrogenicity, general safety and purity standards as required by FDA Office of Biological Standards or corresponding authorities in other countries. Preferred compositions are lyophilized formulations or aqueous solutions.

As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in therapeutic or pharmaceutical compositions is contemplated.

The composition may comprise different types of carriers depending on whether it is to be administered in solid, liquid or aerosol form, and whether it needs to be sterile for such routes of administration as injection. MARV-antibodies can be administered by any method, or any combination of methods as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering protein or polypeptide molecules such as the MARV-antibodies of certain embodiments. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Parenteral compositions include those designed for administration by injection, e.g. subcutaneous, intradermal, intra-lesional, intravenous, intra-arterial, intramuscular, intrathecal or intraperitoneal injection. For injection, the MARV-antibodies may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the MARV-antibodies may be in powder form for constitution with a suitable vehicle, e.g., sterile pyrogen-free water, before use. Sterile injectable solutions are prepared by incorporating the MARV-antibodies in the required amount in the appropriate solvent with various other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi.

Pharmaceutical compositions comprising MARV-antibodies, or fragments thereof, may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

MARV-antibodies may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The pharmaceutical preparation of certain embodiments is a liquid composition, e.g. an aqueous solution. For injection purposes, the use of pure water as solvent is preferred. Other solvents which are suitable and conventional for pharmaceutical preparations can, however, also be employed. In a preferred embodiment, the pharmaceutical compositions are isotonic solutions. Further, there is no need for reconstitution at any stage of the preparation of the liquid solution formulation of these embodiments. The solution is a ready-to-use formulation. The provided anti-viral composition may be produced in an arbitrary unit dose. A unit dose refers to the amount of the active ingredient and the pharmaceutically acceptable carrier contained in each product packaged for use in one or more administrations to a subject, such as a human, and an appropriate amount of such active ingredient and carrier is an amount that may function as an anti-viral when inoculation with the anti-viral composition of the present disclosure is performed one or more times, and such an amount may be determined non-clinically or clinically as understood by those skilled in the art.

Useful delivery vectors for inclusion in the anti-viral compositions include biodegradable microcapsules, immuno-stimulating complexes (ISCOMs) or liposomes. Liposome vectors may also be used for delivery of MARV-antibodies. Such liposome vectors may be unilamellar or multilamellar vesicles, having a membrane portion formed of lipophilic material and an interior aqueous portion. The aqueous portion is used to contain the MARV-antibodies. In general, liposome forming materials have a cationic group, such as a quaternary ammonium group, and one or more lipophilic groups, such as saturated or unsaturated alkyl groups having about 6 to about 30 carbon atoms.

Another aspect pertains to compositions comprising nanoparticles and the disclosed MARV-antibodies or fragments thereof. The nanoparticles can be created from biological molecules or from non-biological molecules. In some cases, the MARV-antibodies are crosslinked to a polymer or lipids on the nanoparticle surface. In embodiments, the MARV-antibodies are adsorbed onto the nanoparticle surface. In some embodiments, the disclosed MARV-antibodies are adsorbed onto the nanoparticle surface and then crosslinked to the nanoparticle surface. In some embodiments, disclosed MARV-antibodies are encapsulated into the nanoparticle. In a specific embodiment, the disclosed antibodies, or fragments thereof, are genetically fused to a nanoparticle gene.

A method of treating, or preventing infection, in a subject is provided that includes administering the disclosed anti-viral composition comprising one or more MARV-antibodies, or fragments thereof, as described herein, to a subject in need thereof. The terms “patient” and “subject” are used interchangeably and include human and non-human animal subjects.

In a non-limiting embodiment, the virus is marburgvirus, and the antiviral composition is able to reduce the severity of symptoms or eliminate the symptoms of, or substantially or completely removing the disease caused by marburgvirus infection. Thus, the anti-viral compositions disclosed herein may be administered prophylactically to a subject, e.g., a human, before infection with marburgvirus, or may be therapeutically administered to subjects after infection with marburgvirus.

The terms “treat/treating/treatment” and “prevent/preventing/prevention” as used herein, refers to eliciting the desired biological response, i.e., a therapeutic and prophylactic effect, respectively. In accordance with the present disclosure, the therapeutic effect includes one or more of a decrease/reduction in the severity of the disease (e.g., a reduction or inhibition of infection), a decrease/reduction in symptoms and disease related effects, an amelioration of symptoms and disease-related effects, and an increased survival time of the affected host, following administration of the anti-viral composition. A prophylactic effect may include a complete or partial avoidance/inhibition or a delay of infection, and an increased survival time of the affected host, following administration of the anti-viral composition.

A method of treating a subject for ebolavirus infection is provided that includes administering the disclosed ebolavirus anti-viral composition to a subject in need thereof. A method of treating a subject is provided that includes administering the disclosed anti-viral composition to a subject in need thereof. Said subjects include any animal that serves as a host for a ebolavirus. Said subject may be an animal under the care of a veterinarian. Said subject may be a mammal. Said subject may be a human.

The disclosed anti-viral compositions may be administered in a number of ways. For example, the disclosed anti-viral composition can be administered orally, intravenously, subcutaneously, transdermally (e.g., by microneedle), intraperitoneally, ophthalmically, vaginally, rectally, sublingually, or by inhalation. The anti-viral composition of the present disclosure may be administered in a controlled release system including, for example, a liposome, a transplantation osmotic pump, a transdermal patch, and the like. In an embodiment, the antiviral compositions may be delivered as a recombinant viral vector engineered to express MARV-antibodies or fragments thereof.

Methods of systemic delivery include those methods known in the art that provide delivery of the active molecule (e.g. the MARV-antibodies) to the circulatory system with distribution throughout the body. Systemic delivery methods include intramuscular, intravenous, subcutaneous, intraperitoneal, and oral. As will be understood, any method of systemic delivery is suitable for use as a means for vaccination. Particularly suitable methods of systemic delivery include intramuscular and intravenous delivery.

In a specific embodiment, the anti-viral compositions are formulated for intranasal administration. Intranasal administration of the anti-viral composition, if used, is generally characterized by inhalation. Compositions for nasal administration can be prepared so that, for example, the MARV-antibodies can be administered directly to the mucosa (e.g., nasal and/or pulmonary mucosa).

Methods for mucosal delivery include those methods known in the art that provide delivery of the composition to mucous membranes. Mucosal delivery methods include intranasal, intrabuccal, and oral. In some embodiments, the administration is intranasal. In these embodiments, the anti-viral composition may be formulated to be delivered to the nasal passages or nasal vestibule of the subject as droplets, an aerosol, micelles, lipid or liquid nanospheres, liposomes, lipid or liquid microspheres, a solution spray, or a powder. The composition can be administered by direct application to the nasal passages or may be atomized or nebulized for inhalation through the nose or mouth.

The dose of the anti-viral composition may be determined by a medical practitioner in consideration of patient characteristics such as age, weight, gender, symptoms, complications, and the incidence of other diseases. Further, the temporal interval of administration and the number of administrations may be determined in consideration of the dosage form that is used, the half-life of the active ingredient in the blood, and the like.

The exact amount of the anti-viral composition required may vary from subject to subject, depending on the species, age, weight and general condition of the subject and its mode of administration and the like. Thus, it is not possible to specify an exact amount for every composition. However, an appropriate amount can be determined by one of skill in the art using only routine experimentation given the teachings herein. For example, effective dosages and schedules for administering the anti-viral compositions may be determined empirically, and making such determinations is within the skill in the art. The dosage ranges for the administration of the anti-viral compositions are those large enough to produce the desired effect in which the symptoms of the disorder are affected. The dosage should not be so large as to cause adverse side effects, such as unwanted cross-reactions, anaphylactic reactions, and the like. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

Toxicity or efficacy of MARV-antibodies components can be determined by standard procedures in cell cultures or experimental animals. Data obtained from cell culture assays and laboratory animal studies can be used in formulating a range of dosage for use in humans. The dosage of such components lies, for example, within a range of administered concentrations that include efficacy with little or no toxicity. The dosage may vary within this range depending upon the dosage form employed and the route of administration utilized.

In still yet another aspect of the present disclosure, a composition and method for detecting a marburgvirus-specific protein, that includes the viral glycoprotein (GP) is provided. The detection composition of the present disclosure serves to detect a marburgvirus-specific glycoprotein, antigen, in a subject sample, and the composition of the present disclosure is able to distinguish marburgvirus-infected and uninfected subjects from each other by bringing the same into contact with a sample and measuring the extent of reaction therebetween. This composition may be useful to distinguish whether a patient with symptoms identical or similar to those of marburgvirus disease is infected with a marburgvirus during the period of risk of onset of marburgvirus mediated disease.

As used herein, the term “sample” refers to a sample in which a marburgvirus-specific antigen, especially a GP antigen, may exist, and includes the blood, serum, plasma, saliva, tears, mucus, nasal mucus and the like.

The antibody/antigen complex may be detected using a detection agent, and the detection agent may be, for example, a secondary antibody binding to the antigen, especially a GP protein. The secondary antibody may be conjugated with a label or an enzyme that provides a detection signal, thus facilitating detection. Label conjugation serves to bind any label capable of providing a detection signal to the antibody. Examples of the label may include radioisotopes such as tritium, iodine (¹³¹I, ¹²⁵I, ¹²³I, ¹²¹I), phosphorus (³²P), sulfur (³⁵S), metals (e.g. ⁶⁸Ga, ⁶⁷Ga, ⁶⁸Ge, ⁵⁴Mn, ⁹⁹Mo, ⁹⁹Tc, ¹³³Xe) and the like, fluorescence substances or fluorophores such as fluorescein isothiocyanate, tetramethyl rhodamine isothiocyanate, substituted rhodamine isothiocyanate, dichlorotriazine isothiocyanate, Alexa or AlexaFluoro, and the like.

Enzyme conjugation serves to bind an enzyme such as peroxidase (POD), alkaline phosphatase, β-galactosidase, urease, catalase, glucose oxidase, lactate dehydrogenase, amylase or a biotin-avidin complex to the antibody, and these enzymes provide a certain detection signal when reacting with a certain substrate. For example, peroxidase shows a purple color when reacting with aminosalicylic acid and hydrogen peroxide or p-phenylenediamine and hydrogen peroxide, alkaline phosphatase shows a yellow color when reacting with dinitrophenylphosphate, and β-galactosidase shows a purple color when reacting with β-nitrophenyl-β-D-galactopyranoside. The label or enzyme may be covalently bonded to the antibody.

Upon detection using the detection agent such as the secondary antibody or the like, the extent of reaction of the secondary antibody with the complex may be measured through a variety of immunoassay methods well known or publicly known in the art, such as enzyme immunoassay, fluorescence immunoassay, radioimmunoassay, luminescence immunoassay, and the like. In a specific embodiment, an enzyme immunoassay, for example an ELISA (enzyme-linked immunosorbent assay), is used.

A further aspect pertains to a diagnostic kit for detecting a marburgvirus-specific antigen, especially a marburgvirus GP antigen. The detection kit of the present disclosure includes the MARV-antibody. The MARV-antibody contained in the kit may be provided in the form of being attached to or detached from a support or may be provided in a dissolved form in a soluble solution or in a lyophilized form.

The diagnostic kit may further include a detection agent for detecting a complex of the marburgvirus-specific antigen, especially the Marburgvirus GP. The detection agent may be a secondary antibody conjugated with the label or enzyme described above.

Furthermore, the diagnostic kit may further include a carrier, a washing buffer, a diluted sample solution, an enzyme substrate, and a reaction stop solution, and may also include instructions to teach the method of use, including a method of analysis of the results, etc.

Still a further aspect pertains to a diagnostic method of detecting a marburgvirus-specific antigen, especially a marburgvirus GP, in a bio-sample. The method includes (a) contacting a sample with the MARV-antibody composition for detecting a marburgvirus-specific antigen, and (b) detecting the complex. In an embodiment, the bio-sample in step (a) is a nasal swab.

Also, in the diagnostic method, the detecting the complex in step (b) includes reacting a secondary antibody conjugated with a label or an enzyme capable of providing a detection signal with the complex and measuring the extent of reaction with the complex. The extent of reaction of the secondary antibody with the complex may be measured through enzyme immunoassay, fluorescence immunoassay, radioimmunoassay, luminescence immunoassay, etc., as described above. In a specific embodiment, an ELISA (enzyme-linked immunosorbent assay) is used.

In another aspect of the embodiment, an article of manufacture (e.g., a kit) containing materials useful for the treatment of marburgvirus as described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle).

The label or package insert indicates that the composition is used for treating the condition of choice. The article of manufacture may comprise a container with a composition contained therein, wherein the composition comprises MARV-antibodies.

Kits in certain embodiments may further comprise a package insert indicating that the compositions can be used to treat a particular condition. Alternatively, or additionally, the kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The anti-viral compositions may, if desired, be presented in a pack or dispenser device which may contain one or more-unit dosage forms containing the MARV-antibodies. The pack may for example include metal or plastic foil, such as a blister pack. The pack or dispenser device may be accompanied by instructions for administration to subjects, especially humans. Associated with such container(s) can be a notice in the form prescribed by a governmental agency regulating the manufacture, use or sale of pharmaceuticals or biological products, which notice reflects approval by the agency of manufacture, use or sale for human administration. Thus, a kit is provided that includes the MARV-antibodies, as described herein. In one specific aspect the kit further includes instructions for the treatment and/or prophylaxis of marburgvirus infection.

Unless otherwise defined, all technical and scientific terms used herein have the same meaning as commonly understood by one skilled in the art. Although methods and materials similar to or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Example 1

### Materials and Methods

Virus-like particles composed of filovirus GP, NP, and VP40 of species MARV (Musoke), SUDV (Yambio), and EBOV (Mayinga) and recombinant GPΔTM and GPΔMuc antigens corresponding to species MARV (Angola), SUDV (Yambio), and EBOV (Mayinga) were purchased from an outside vendor (Integrated Biotherapeutics, Gaithersburg, MD) (30). The GPΔTM antigens included full GP ectodomains covering residues 1-627 of EBOV and SUDV GP, and residues 1-636 of MARV GP. GPΔMuc antigens were comprised of GP residues 1-311 fused to residues 464-637 for EBOV and GP residues 1-313 fused to residues 474-640 for SUDV. Recombinant BDBV and RAVV glycoproteins were expressed in HEK293 cells, as previously described (38).

GP full-length sequence identity matrices were calculated in Bioedit using orthomarburgvirus strains MARV Musoke YP_001531156.1, MARV Angola Q1PD50.1, and RAVV Ravn YP_009055225.1, and orthoebolavirus strains EBOV Mayinga AAN37507, SUDV Yambio ABY75325, and BDBV AGL73460.1.

Three Rhesus macaques (two females and one male) of the species Macaca mulatta of Chinese origin were immunized intramuscularly 4 times at 2-week intervals. Animals were ˜5 years of age and weighed between 4.04 and 5.86 kg. MARV VLP prime immunizations were administered at 1 mg doses. MARV and SUDV VLP bivalent boosts were administered at 0.5 mg doses for each species. All recombinant GP ectodomain antigens were administered at 100 mcg for each species. All immunizations were formulated in 0.5 mL TiterMax Gold adjuvant (Sigma-Aldrich Inc., St. Louis, MO). Bleeds were conducted on day 7 after each inoculation to collect serum and peripheral blood mononuclear cells (PBMCs).

Nunc MaxiSorp 96-well ELISA plates (Thermo Fisher Scientific Inc., Waltham, MA) were coated with filovirus GPΔMuc proteins at 4° C. overnight. Plates were washed in PBS pH 7.4 containing 0.05% Tween 20 and then blocked in PBS pH 7.4, 5% fetal bovine serum, and 2% non-fat dry milk powder for 1 hour at room temperature. The plates were washed and then incubated with fivefold serial dilutions of either monoclonal antibody or serum starting at 10 μg/mL or 1/10 dilution, respectively, for 1 hour. Plates were washed and a 1/2,500 dilution of horseradish peroxidase-conjugated goat anti-human secondary antibody (Jackson Immunoresearch, West Grove, PA) in blocking buffer was added for 1 hour. After washing, ELISAs were developed with TMB ELISA substrate solution (Bio-Rad Laboratories, Inc., Hercules, CA) and stopped using 1N sulfuric acid. Plates were read at an absorbance of 450 nm.

Competition ELISAs were undertaken by coating half-area ELISA plates (Greiner Bio-One, Monroe, NC) with 1 μg/mL of the benchmark antibodies at 4° C. overnight. The next day, plates were washed in a wash buffer containing PBS pH 7.4 supplemented with 0.05% Tween 20 and then blocked in PBS pH 7.4, 5% fetal bovine serum, and 2% non-fat dry milk powder for 1 hour at room temperature. During this time in a separate non-binding U well shape plate (Greiner Bio-One, Monroe, NC) competing antibody was diluted to a final concentration of 5 μg/mL in blocking buffer and added to 2 μg/mL GP that was biotinylated through a fused Avi-tag (Avidity, Aurora, Colorado) and incubated for 1 hour at room temperature. The GP antibody mixture was then added to ELISA plates coated with capture antibody and incubated for 1 hour at room temperature. Plates were washed in wash buffer and then incubated with a 1:10,000 dilution of goat anti-biotin antibody (Thermo Fisher Scientific, Waltham, MA) in PBS pH 7.4 supplemented with 0.05% tween-20. The ELISA was developed as described above.

The generation of Murine Leukemia Virus (MLV)-based pseudoviruses with different filovirus GPs was carried out as previously described (42). Briefly, codon-optimized full-length genes of wild-type EBOV GP (GenBank: AAN37507.1), SUDV GP (GenBank: ALL26375.1), BDBV GP (GenBank: AGL73460.1), MARV GP (GenBank: YP_001531156.1), and RAVV GP (Genbank: Q1PDC7.1) were synthesized and constructed into a pCDNA3.1(−) expression vector using XbaI and HindIII (GenScript, Piscataway, NJ). The pseudoviruses were then produced by co-transfection of the human embryonic kidney 293T (HEK 293T) cells with the MLV Gag-Pol packaging vector (kindly provided by Dr. Jonathan K. Ball, the University of Nottingham), the Luciferase reporter plasmid (kindly provided by Dr. Jonathan K. Ball), and the GPs of five filoviruses constructs using Lipofectamine 3000 (Thermo Fisher Scientific, Waltham, MA) by following the manufacturer's protocols. No-envelope control (empty plasmid) was used as a negative control in the experiments. After 6 hours, the medium was replaced by fresh DMEM with 10% FBS. At 48 hours and 72 hours after transfection, the culture supernatants containing filoviral pseudoviruses were harvested, passed through 0.45-μm pore-size filters, and used to infect target cells. Luciferase activity was detected using BrightGlo (Promega, Madison, WI) and expressed as relative light units (RLU) to determine the dilution used in neutralization assays.

Production of Vesicular Stomatitis Virus (VSV)-based filovirus GP pseudoviruses was performed according to the manufacturer's instructions (Kerafast, Boston, MA). Briefly, HEK293T cells transfected with respective full-length filovirus GPs were transduced with VSVΔG-G for 2-4 hours (43). Cells were washed twice with PBS 7.4 and grown in DMEM containing 1.5% FBS and 1% Pen-Strep for 24 hours at 37° C. and 5% CO₂. VSVΔG-GP supernatants were collected and centrifuged for 10 minutes at 300×g and passed through a 0.45-μm filter.

To test animal sera for MLV or VSV-based pseudovirus neutralization, Vero E6 cells (ATCC, Manassas, VA) were pre-seeded into 96-well plates at minimum densities of 1×10⁴ cells per well and grown overnight at 37° C. and 5% CO₂ in DMEM containing 10% FBS and 1% Pen-Strep. The next day, pseudoviruses were incubated with defined concentrations of heat-inactivated serum at serial dilutions for 1 hour at 37° C. and then added to each well. For MLV pseudovirus assays, the plates were incubated in a CO₂ incubator at 37° C. for 5 hours, followed by replacement of the mixtures with fresh medium and continued incubation for 72 hours at 37° C. For VSV pseudovirus assays, the plates were incubated for 1 hour at 37° C., 5% CO₂ followed by the addition of an equal volume DMEM containing 5% FBS and 2% Pen-Strep, and continued incubation for 24 hours. To subsequently measure the degree of viral entry, luciferase activity in cell lysates was measured with the Bright-Glo Luciferase Assay System according to the manufacturer's instructions (Promega, Madison, WI). Luciferase levels were measured using a FLUOstar Omega plate reader (BMG Labtech, Cary, NC) or a Tecan Spark 10M Plate reader (Tecan, Männedorf, CH). 50% and 80% inhibitory dilution (ID50 and ID80, respectively) titers were calculated as the serum dilution that led to a 50% or 80% reduction in relative light units (RLU) compared with pseudoviruses in control wells. ID50 and ID80 values were calculated through a dose-response curve fit with nonlinear regression plots using GraphPad Prism. All experiments involving the use of pseudoviruses were performed under biosafety level 2 conditions.

The inhibition of the animal sera-mediated neutralization of MARV infection was tested using a neutralization inhibition assay (44) in which the macaque NHP1 sera (study day 49 and pre-immune) were preincubated for 30 minutes with purified RAVV GPΔMuc at concentrations of either 2.5 μg/mL or 25 μg/mL before the addition of the MARV pseudoviruses. After incubating for 1 hour at 37° C., the mixtures were then added to the 96-well plates of Vero E6 cells and further incubated for 5 hours before replacing them with fresh medium. With another 72-hour incubation, the luciferase activity was measured using the same method as described in the neutralization assays above. The inhibition effect of recombinant GP on MARV pseudovirus neutralization was reported as the change between the serum ID50 with or without the presence of the tested GP competitor. The neutralization inhibition efficiency was calculated based on the following calculation: [(percentage of neutralization w/o GPs-percentage of neutralization with GPs)/(percentage of neutralization w/o GPs)]×100. PBS was used as the negative control in the experiment.

Macaque monoclonal antibodies were isolated by single B-cell cloning as previously described (45,-47). In brief, macaque PBMCs were thawed and resuspended in staining media made up of RPMI 1640 supplemented with 10% fetal calf serum (FCS) at 37°. Cells were then washed in 10 mL staining media containing DNase I (Roche, Basel, Switzerland) and then resuspended in 100 μL of staining media containing 4 μg/mL of biotinylated RAVV GPΔMuc conjugated to streptavidin PE and 4 μg/mL of biotinylated RAVV GPΔMuc conjugated to streptavidin APC and incubated for 20 minutes. This was followed by the addition of a cocktail of CD3 APC-Cy7, CD8 APC-Cy7, Aqua Dead, CD14 Qdot 605 (BV605), IgM PE-Cy5, CD27 PE-Cy7, IgG FITC and CD20 PE-Alexa Fluor 700. Cells were gated for RAVV GPΔMuc double-positive B cells with a phenotype CD27+, IgG+, CD20+, Aqua Dead−, CD3−, CD8−, IgM− and sorted at single-cell precision on a BD FACSAria II (46). Individual cells were sorted directly into lysis buffer and then subjected to a reverse transcription-polymerase chain reaction (RT-PCR) using Superscript IV, as per the manufacturer's guidelines (Thermo Fisher Scientific, Waltham, MA). Nested PCR using HotStarTaq (Qiagen, Hildent, Germany) was then used to amplify individual heavy and lambda/kappa light chains from the RT-PCR product. Heavy and light chain pairs were identified by agarose gel electrophoresis and sequenced by Sanger sequencing (Eurofins Genomics, Louisville, KY).

Antibody variable heavy and light chain regions were synthesized by gene synthesis with appended N-terminal signal sequences (Genscript Biotech, Piscataway, NJ) and subcloned into human IgG1 or lambda or kappa light-chain-based PCDNA3.1 mammalian expression plasmids. Plasmids were co-transfected into HEK-293F cells (ATCC, Manassas, VA) in FreeStyle Media using 293Fectin for transient protein expression (Thermo Fisher Scientific, Waltham, MA). Secreted IgGs were purified from cell supernatants with Protein A resin (Roche, Basel, Switzerland). IgGs were eluted at low pH using Protein A elution buffer (Thermo Fisher Scientific, Waltham, MA) and neutralized with Tris base pH 9.0. The IgGs were further purified by size exclusion chromatography (SEC) using an S200 column (Cytiva Lifesciences, Marlborough, MA) in PBS pH 7.4.

RAVV GPΔMuc fused with Hisx8, Strep II and Avi tags and a fibritin foldon trimerization domain was expressed in HEK293S GNTI^{-/-} cells (ATCC, Manassas, VA) using 293fectin transfection reagent and FreeStyle Media (Thermo Fisher Scientific, Waltham, MA). Supernatants were purified using Streptactin XT Resin purification (IBA Lifesciences, Göttingen, Germany). The GP protein was further purified by SEC Superdex 200 HiLoad 16/600 column in 150 mM NaCl, 2.5 mM Tris-Cl pH 7.5, and 0.02% NaN3. GP was biotinylated by the Avi tag (Avidity, Aurora, Colorado) and exchanged into PBS 7.4.

Peptide competitions were undertaken by incubating RAVV GPΔMuc at 200 ng per well in Nunc MaxiSorp 96-well ELISA plates (Thermo Fisher Scientific, Waltham, MA) overnight at 4° C. The plates were blocked as described above. In separate non-binding U-well-shaped plates (Greiner Bio-One, Monroe, NC), 200 ng of each of the overlapping 15-mer peptides across either GP1 or GP2 was incubated individually with 0.4 μg/mL of IgG for 1 hour at room temperature. 100 μL of each IgG peptide mixture was added to the plate with GP and incubated for 1 hour. Plates were washed and a horseradish peroxidase-conjugated goat anti-human secondary antibody (Jackson ImmunoResearch, PA) was added at a 1:2,500 dilution. Plates were washed and developed as above. Direct pepscan analysis was undertaken by binding 200 ng of each overlapping peptide to Nunc MaxiSorp 96-well ELISA plates (Thermo Fisher Scientific, Waltham, MA) overnight at 4° C. Plates were washed and blocked as above, and 0.4 μg/mL of IgG was then added to each well for 1 hour at room temperature. Plates were washed, and secondary antibodies were added as described above. Finally, plates were washed and developed as described above.

Mini-PROTEAN TGX gels were run and transferred to a PVDF or nitrocellulose membrane using the Turbo Blot protocol for mini-PROTEAN TGX gels (Bio-Rad Laboratories, Hercules, CA). The membranes were blocked for 5 minutes with 5% skim milk powder. The PVDF/nitrocellulose membranes were then divided into strips of one lane, each containing GP1 and GP2. The strips were placed in separate primary stain solutions with each antibody at 1 μg/mL. After staining on a platform rocker for 1 hour, the strips were washed three times for 5 minutes with TBST. All strips were then stained separately with a 1:5,000 dilution of goat anti-human IgG for 1 hour. After the secondary staining, the strips were again washed three times with TBST for 5 min. The strips were then placed together and developed by enhanced chemiluminescence (ECL) (Thermo Fisher Scientific, Waltham, MA) and imaged on a ChemiDoc imager (Bio-Rad Laboratories, Hercules, CA).

Negative staining was performed following the optimized negative staining (OpNS) protocol as described (48). Optimized negative staining: a high-throughput protocol for examining small and asymmetric protein structure by electron microscopy (48). Briefly, complexes were diluted to 0.01 mg/mL and immediately applied to EM grids (Electron Microscopy Sciences #CF200-Cu). Grids were then incubated for 1 minute, blotted with filter paper, washed three times with water as described, and stained with fresh 1% uranyl formate solution for 30 s as described. The staining solution was then blotted with filter paper and grids were dried in a desiccator overnight prior to imaging.

Imaging was performed using a Talos Arctica (200 kV) system (Thermo Fisher Scientific) equipped with a Falcon 3EC detector. A nominal magnification of 73,000× was used, corresponding to a pixel size of 1.38 Å. Dose-fractionated movies were collected with a total dose of about 120 e/Å², and motion correction was performed using RELION (49). Particle picking was done using crYOLO followed by 2D classification in RELION (49, 50).

HEK293T cells were seeded at approximately 70% confluence in 96-well plates and transfected with MARV Musoke GP using Lipofectamine 3000 (Thermo Fisher Scientific, Waltham, MA). The following day VSVΔG-G virus was added at 40 μL per well and incubated for 2 hours at 37° C. and 5% CO₂. Cells were then washed with DMEM containing 10% FBS, and antibodies pre-diluted at 50 μg/mL in 100 μL DMEM with 10% FBS added and incubated for 6 hours at 37° C. and 5% CO₂. The sample was harvested by diluting 10 μL of supernatant into 90 μL DMEM supplemented with 10% FBS followed by centrifugation at 1,000×g for 5 minutes to remove cells. 90 μL of each sample was then added to VeroE6 cells seeded the prior day at 20,000 cells/well in 96-well plates. Plates were incubated overnight at 37° C. and 5% CO₂ and the following day were developed using BrightGlo (Promega, Madison, WI) following the manufacturer's protocols. Virus titers were measured in relative luminescence units (RLU). Relative titers in the presence versus absence of antibodies were calculated using the formula: (RLU [with antibody]/RLU [without antibody])*100%. A two-tailed unpaired t-test was used in Graphpad Prism to assess statistical significance relative to the no-antibody controls. RESULTS

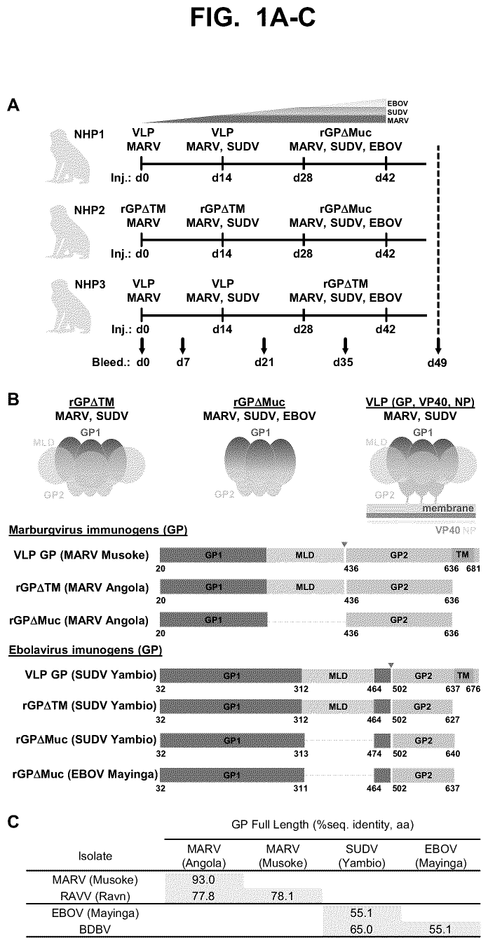

Three Rhesus macaques of Chinese origin (two females and one male) were immunized with multivalent regimens made up of MARV, SUDV, and EBOV GP-based immunogens (FIG. 1A). To restrain possible immunodominance of EBOV GP (30) and enhance immune responses primarily against MARV GPs but also against SUDV GPs, a multivalent prime-boost approach weighted with MARV and SUDV immunogens was employed. All animals were primed exclusively with MARV-based immunogens, followed by bivalent boosts with MARV+SUDV immunogens, followed by two trivalent boosts with MARV+SUDV+EBOV immunogens (FIG. 1A). Animals were immunized a total of four times at 2-week intervals (study days 0, 14, 28, and 42) and bleeds were taken at day 7 after each vaccination, a timepoint shown to have a high frequency of antigen-specific plasmablasts (51, 52) (FIG. 1A). Immunogens included VLPs made up of GP, VP40, and NP, and recombinant GP ectodomains either with or without intact mucin-like domains (GPΔTM or GPΔMuc, respectively) (FIG. 1B). Recombinant glycoprotein immunizations were formulated in Titermax Gold adjuvant, a water-in-oil emulsion. While all SUDV and EBOV immunogens were based on the Yambio and Mayinga isolates, respectively, MARV immunogens were based on Musoke for the VLPs and Angola for the recombinant GPs, which further increased the antigenic breadth of immunized MARV variants. Intra-genus full-length GP sequence diversity for the corresponding immunogens was ˜7% for the orthomarburgvirus antigens and ˜45% for the orthoebolavirus antigens (FIG. 1C).

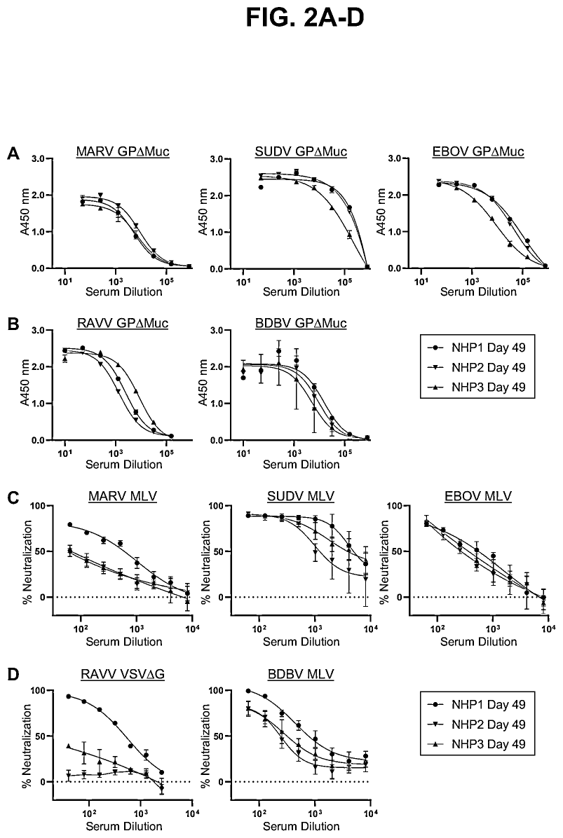

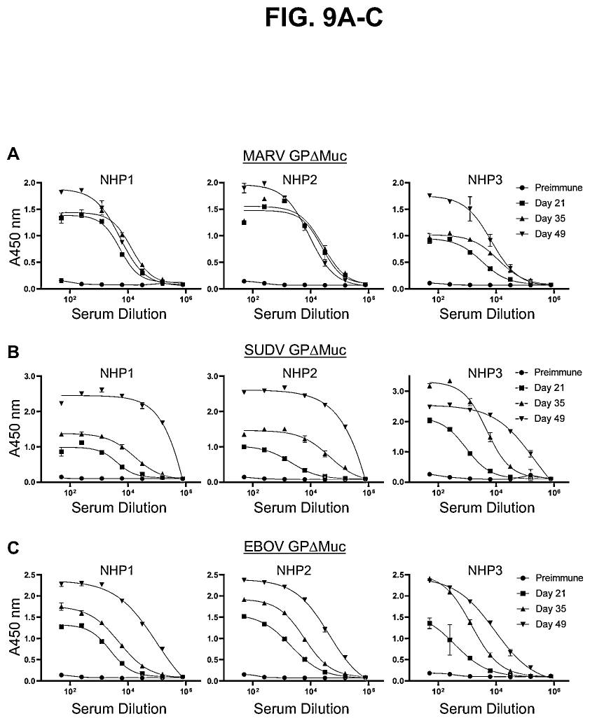

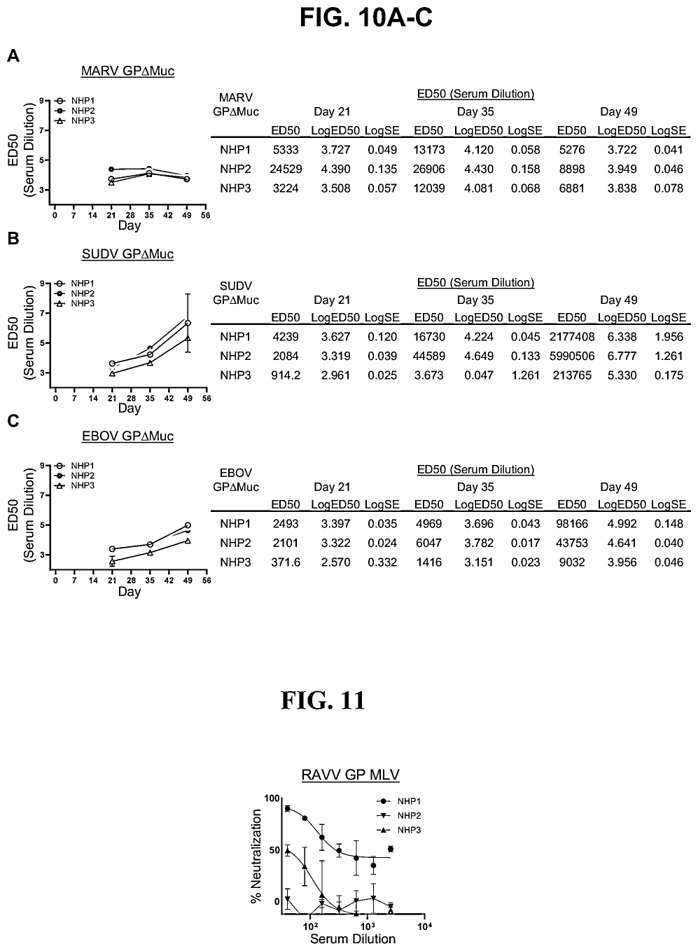

To assess serum IgG antibody binding titers elicited over the course of the study against autologous filovirus GP species, serum bleeds taken 7 days after the first, second, and final boosts (study days 21, 35, and 49) were tested for binding to recombinant MARV, SUDV, and EBOV GPΔMuc by ELISA. Serum IgG binding titers against all three autologous species were detected in all animals (FIG. 2; FIG. 9). Serum from day 49 terminal bleeds yielded 50% effective dilutions (ED50s) of binding to MARV, EBOV, and SUDV GPΔMuc that ranged from ˜5.8 to 8.9×10³, ˜9.0 to 98×10³, and 2.1 to 60×10⁵, respectively, with binding responses in animal NHP3 lagging behind those in NHP1 and NHP2 (FIG. 2A). Over the course of the study, responses against SUDV and EBOV GPΔMuc increased after the second and third boosts while those against MARV GPΔMuc were generally less responsive to these boosts (FIG. 9, FIG. 10).

Since one of the objectives of the multivalent immunization approach was to induce immunological breadth against conserved regions within filovirus GPs, serum antibody recognition of heterologous filovirus GPs was also assessed. Terminal bleed day 49 serum from each of the three immunized macaques was tested by ELISA for recognition of heterologous RAVV and BDBV GPΔMuc proteins, which differ in sequence from their autologous full-length counterparts by up to 22.2% and 44.9%, respectively (FIG. 1C). The presence of IgG binding titers against RAVV and BDBV GPΔMuc was detected in sera from all three animals, with serum dilution ED50s ranging from 1.4 to 8.5×10³ and 5.0 to 14×10³, respectively (FIG. 2B). Serum reactivity against EBOV GPΔMuc was detected in day 21 serum from all three animals, a time point in the study preceding any EBOV GP immunizations, indicating the presence of heterologous binding titers against this species as well (FIG. 9, FIG. 10). Taken together, the data confirmed that the heterologous prime-boost immunization approach employed in the study led to the successful elicitation of antibodies with both autologous and heterologous binding breadth.

Terminal bleed serum (day 49) from all three animals was next tested for neutralization of Murine Leukemia Virus (MLV)-based viruses pseudotyped with autologous MARV Musoke, SUDV Boniface, or EBOV Mayinga GP. Animal NHP1, which received a MARV VLP prime, a MARV+SUDV VLP-based boost, followed by two trivalent boosts with MARV, SUDV, and EBOV GPΔMuc proteins, exhibited the highest overall IgG neutralization titers observed in the study against all three autologous viral species (FIG. 2C). Neutralization ID50s in this animal were observed at ˜7.1×10², ˜5.6×10³, and ˜2.0×10³, against MARV, SUDV, and EBOV pseudoviruses, respectively (FIG. 2C). Although neutralization titers against SUDV and EBOV were also observed in serum from animals NHP2 and NHP3, neutralizing titers against MARV in these animals were lower than those observed in NHP1 despite the presence of nearly equivalent levels of MARV GPΔMuc binding titers (FIG. 2A, FIG. 2C).

To determine whether heterologous neutralization breadth was induced in the animals, terminal bleed day 49 serum from each animal was tested for neutralization of heterologous BDBV and RAVV pseudoviruses. As shown in FIG. 2D, neutralizing antibody titers against heterologous BDBV were observed in all three animals, with animal NHP1 serum yielding the highest heterologous neutralization potency of the three animals. Heterologous neutralizing titers against RAVV were tested using both VSV- and MLV-based pseudoviruses, revealing neutralizing titers mainly in NHP1 serum, with reduced or absent neutralizing responses in NHP3 and NHP2 sera, respectively (FIG. 2D; FIG. 11).

Taken together, these results indicated that the most pronounced neutralizing responses, against both autologous and heterologous viruses, were induced in NHP1, prompting us to further investigate the induced mAbs in this animal.

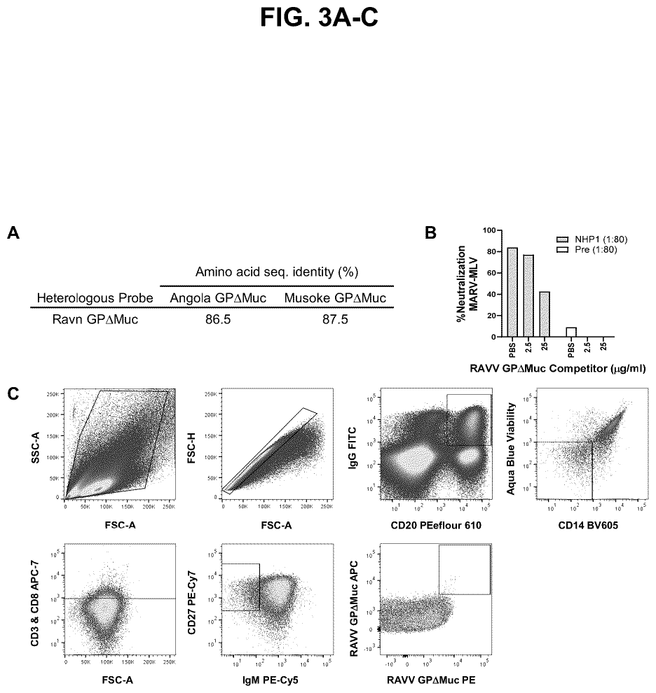

To isolate cross-reactive antibodies, a recombinant heterologous GP probe to select for cross-orthomarburgvirus reactive memory B cells from peripheral blood mononuclear cells (PBMCs) of animal NHP1 was developed. Toward this end, a fibritin foldon-trimerized heterologous RAVV GPΔMuc protein shown above to be recognized by NHP1 serum was utilized (FIG. 2B), which diverged in amino acid sequence from autologous Musoke and Angola MARV GPΔMuc by ˜13% (FIG. 3A). To validate its use as a probe, its efficacy as a competitor in MARV-MLV pseudovirus neutralization assays was assessed, to gauge if it could be bound effectively by NHP1 serum heterologous neutralizing antibodies. Although the addition of the RAVV GPΔMuc probe at 2.5 μg/mL concentration to NHP1 serum prior addition to pseudoviruses and target cells was not sufficient to compete away serum neutralization, when added at 25 μg/mL it successfully reduced serum neutralization by ˜60% (FIG. 3B). These results confirmed the presence of heterologous cross-orthomarburgvirus reactive neutralizing antibodies in NHP1 serum and validated the use of RAVV GPΔMuc as a heterologous probe for B-cell sorting.

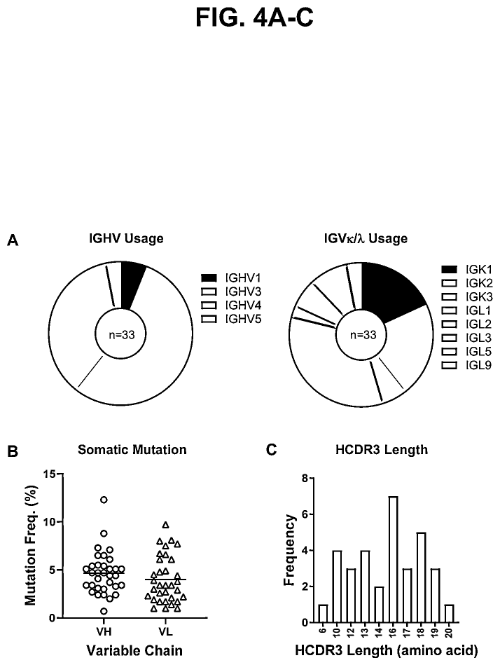

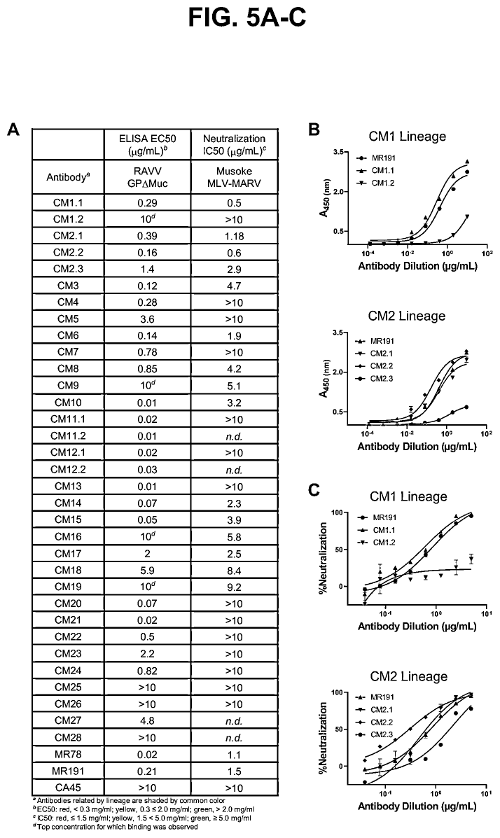

To isolate double-positive RAVV GPΔMuc reactive B cells, NHP1 terminal bleed (day 49) peripheral blood mononuclear cells (PBMCs) were stained with avi-tag biotinylated trimerized RAVV GPΔMuc protein conjugated with two types of fluorescently labeled streptavidin, APC and PE, along with a cocktail of reagents targeting memory B-cell surface markers. The multi-color staining approach ensured the selection of B cells that were of the phenotype IgG⁺IgM⁻CD20⁺CD14⁻CD3⁻CD8⁻CD27⁺, RAVV GP⁺⁺ (46, 53) (FIG. 3C). Of the ˜480 B cells that were sorted into 96-well plates, the first 96-well plate was utilized to recover an initial panel of monoclonal antibodies through nested PCR amplification of heavy and light chain antibody variable regions, as previously described (46, 53). 58 out of 96 wells yielded successful amplification of both heavy and light chain antibody products that were subsequently sequenced. Based on a variety of sequence features, including sequence fidelity and completeness, immunogenetic diversity, the presence of lineage mates, HCDR3 loop length, and degree of somatic hypermutation, 34 mAb heavy and light chain pairs were selected for experimental characterization. The selected mAb sequences represented diverse immunogenetic backgrounds, corresponding to roughly 10 IGHV and 21 IGLV genes (FIG. 4A). A majority of the heavy chains were of VH3-background (FIG. 4A). Rates of somatic hypermutation ranged from 0.7% to 12.3% and 1.0% to 9.7% for heavy and light chains, respectively, while heavy chain CDR3 loop lengths ranged from 6 to 20 amino acids (FIG. 4B; FIG. 4C). A majority of the antibodies in the panel represented independent clonotypes, although nine variants belonged to one of four shared lineages (FIG. 5A). For experimental characterization, the heavy and light chain variable regions of the selected 34 mAbs were synthesized and subcloned into human IgG1 expression vectors for transient expression in HEK293 cells. Out of the 34 mAbs, 33 expressed to sufficient levels to permit further study.

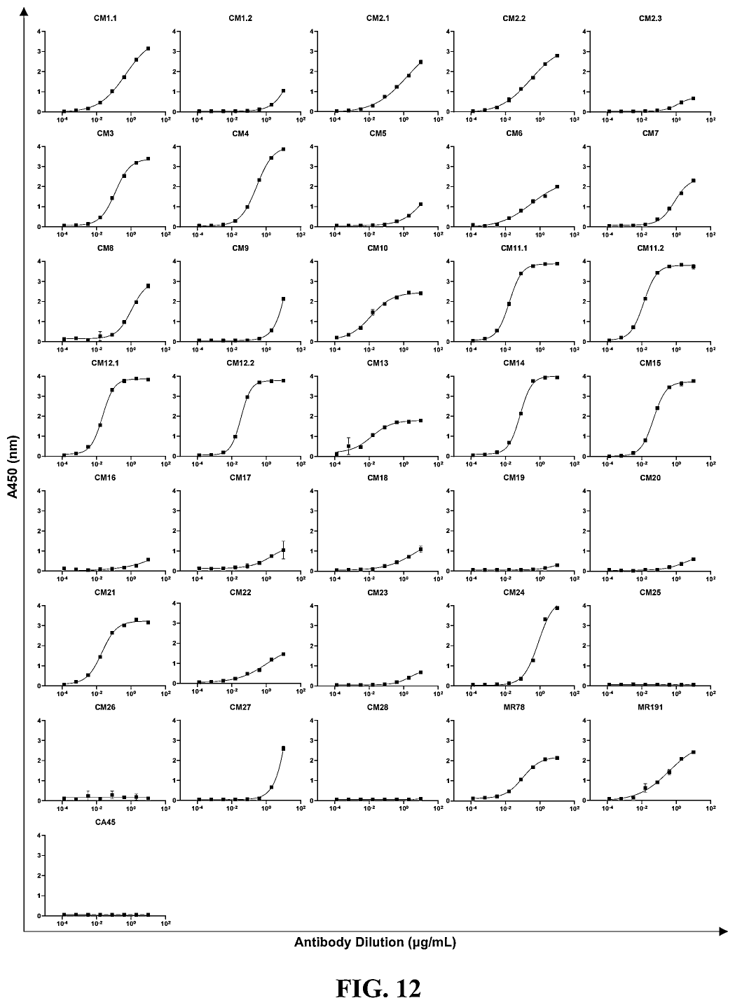

The binding of the 33 expressed mAbs to RAVV GPΔMuc by ELISA was assessed, alongside orthomarburgvirus GP-specific antibodies MR78 and MR191 and orthoebolavirus GP-specific antibody CA45 as controls (11, 31). 28 of the mAbs (representing 23 lineages) bound RAVV GPΔMuc with EC50 values that ranged from 0.01 to 10 μg/mL, confirming that the B-cell sorts led to successful isolation of orthomarburgvirus GP-specific mAbs (FIG. 5A; FIG. 12). Indeed, some of the antibodies bound with EC50 values that were commensurate or better than those observed for control antibodies MR78 and MR191 (FIG. 5A; FIG. 12).

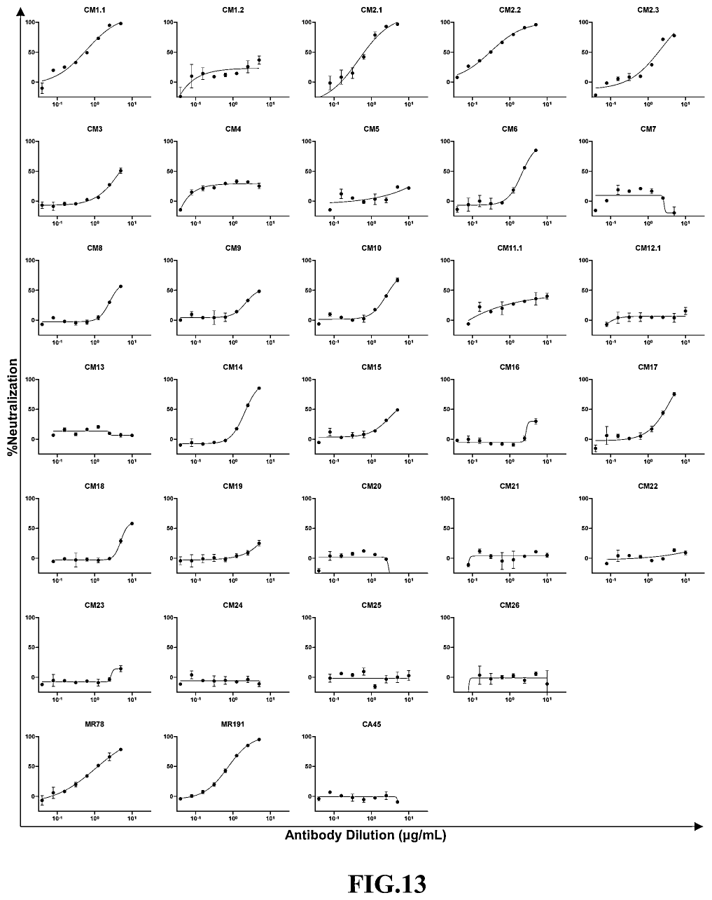

Using a maximum antibody concentration of 10 μg/mL, the 28 GP-reactive mAbs were next tested for the capacity to neutralize MLV-MARV Musoke pseudoviruses (54). 16 of the 28 antibodies tested (˜57%) exhibited neutralization of MLV-MARV to different degrees, with neutralization IC50 values ranging from 0.5 to 9.2 μg/mL (FIG. 5A; FIG. 13). Two of the antibody lineages, CM1 and CM2, exhibited the most potent neutralization observed in the panel with IC50s that ranged from 0.5 to 1.18 μg/mL, on par with IC50s obtained for the MR191 and MR78 controls (FIG. 5A; FIG. 5C; FIG. 13) (11). While some of the variants that belonged to the CM1 and CM2 lineages exhibited weak or undetectable neutralization, namely mAbs CM1.2 and CM2.3, such differences correlated with differences in binding capacity to recombinant GP by ELISA (FIG. 5A-C; FIG. 12-13). In contrast to their lineage mates, mAbs CM1.2 and CM2.3 also expressed at lower levels and were prone to proteolytic cleavage, consistent with potential biochemical instability. Nonetheless, the results confirmed that a majority of the mAbs in the panel effectively recognized heterologous RAVV GPΔMuc, with two of the lineages exhibiting highly potent MARV pseudovirus neutralization.

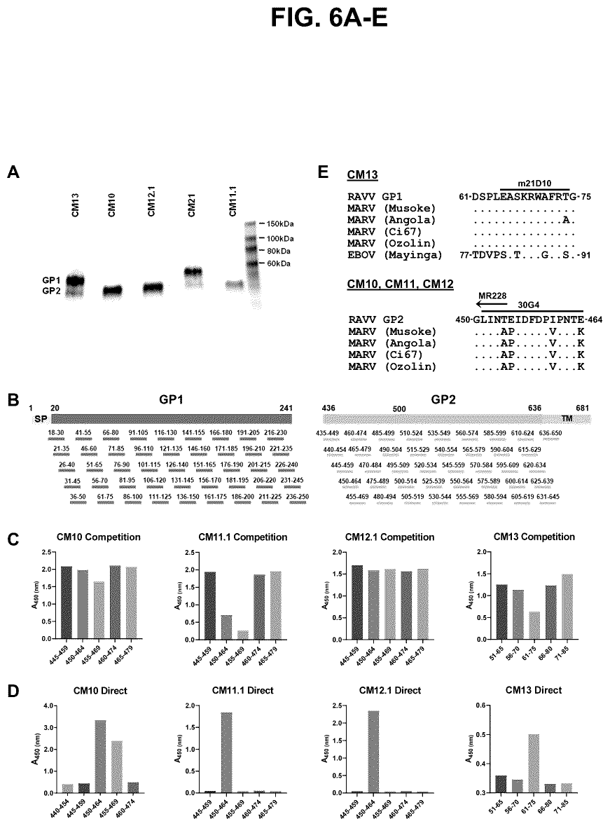

Prior to undertaking overlapping pepscan analysis for epitope mapping, it was assessed whether any of the GP-reactive antibodies in the panel could recognize contiguous, non-conformational epitopes on RAVV GPΔMuc. Toward this end, RAVV GPΔMuc protein was applied to a denaturing SDS-PAGE gel and subjected to standard Western blotting procedures, using each individual GP-reactive antibody as a probe. Five of the tested mAbs gave detectable signals by Western blot analysis (FIG. 6A). Two mAbs, CM13 and CM21, reacted with a band corresponding to the size of GP1, while the remaining three mAbs, CM10, CM11.1, and CM12.1, targeted a band corresponding to the size of GP2 (FIG. 6A).

To further map the epitopes of these five Western-blot reactive mAbs, a panel of overlapping 15-mer peptides covering the sequences of GP1 and GP2 of RAVV GPΔMuc was generated, and both competition and direct ELISA binding analyses were conducted (FIG. 6B-D). For mAbs CM10, CM11.1, and CM12.1, which were predicted to target the GP2 subunit, the analysis focused on binding to 46 overlapping peptides covering the GP2 ectodomain, spanning residues 435-650. To assess whether any of the 46 overlapping GP2 peptides could successfully compete for mAb recognition of RAVV GPΔMuc, each mAb was individually incubated with each peptide and then added the mixture to ELISA wells coated with RAVV GPΔMuc. These assays revealed that peptides 450-464 and 455-469 within the GP2 N-terminus (or “wing”), a region previously shown to be targeted by protective antibodies, successfully competed for CM10 and CM11.1 recognition of RAVV GPΔMuc (FIG. 6C and FIG. 6E) (17, 18). None of the peptides effectively competed for CM12.1 mAb recognition of RAVV GPΔMuc (FIG. 6C). To further verify CM10 and CM11.1 recognition of the GP2 N-terminus, and to also map the epitope of mAb CM12.1, direct ELISA binding analyses using the same set of overlapping peptides spanning the GP2 N-terminus (residues 440-479) was conducted. mAbs CM10 and CM11.1 both bound peptide 450-464 directly, while mAb CM10 bound peptide 455-469 as well. Despite the inability of peptide 450-464 to effectively compete with RAVV GPΔMuc for CM12.1 recognition, direct binding of CM12.1 to peptide 450-464 was detected (FIG. 6C and FIG. 6D). The results thus indicate that mAbs CM10, CM11.1, and CM12.1 all target an epitope within the GP2 N terminus, one that overlaps with epitopes of previously reported protective mAbs isolated from natural infection and animal immunizations (FIG. 6E) (11, 17, 18, 55).

To map the epitopes of GP1 Western-blot reactive mAbs CM13 and CM21, a similar strategy was employed but utilized a set of overlapping GP1 peptides instead (FIG. 6B). 45 overlapping 15-mer peptides spanning GP1 ectodomain residues 18-250 were used as competitors for CM13 and CM21 binding to RAVV GPΔMuc (FIGS. 6B and C). Binding of CM13 to RAVV GPΔMuc was competed ˜50% by a peptide spanning GP1 residues 61-75, although direct recognition of this peptide was weak (FIGS. 6C and D). It was noted that peptide 61-75 lies in the vicinity of the predicted RBR on GP1, and partially overlaps with the epitope of a previously reported pan-filovirus reactive murine antibody, m21D10, one that was also isolated from multivalent immunization (FIG. 6E) (29). In contrast to CM13, none of the 45 overlapping 15-mer GP1 peptides competed with mAb CM21 for binding to RAVV GPΔMuc, nor were any recognized by direct ELISA (not shown), indicating other means will be necessary to map its epitope

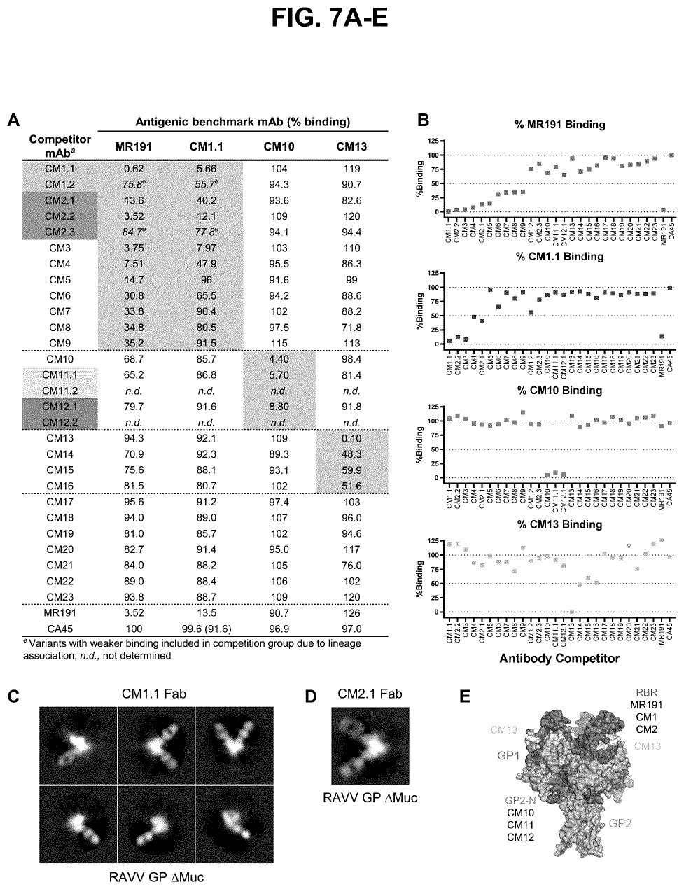

To further classify the antigenic targets of the antibodies in the panel, GP binding competition analyses were performed to define antigenic competition groups. Four antibodies were selected as antigenic benchmarks for recognition of RAVV GPΔMuc against which all antibodies in the panel were tested as competitors. Benchmark mAbs included CM10 and CM13 that were mapped above to continuous epitopes on GP2 and GP1, respectively, along with two potent MARV neutralizing antibodies, CM1.1 from the present study and antibody MR191, a previously reported RBR-directed nAb (FIG. 7A-B) (11). The binding competition assay entailed pre-incubation of each GP-reactive antibody in the panel with biotinylated RAVV GPΔMuc for 1 hour followed by the addition of the complex to ELISA plates pre-coated with each of the four antigenic benchmark antibodies. The degree to which the benchmark antibodies could capture biotinylated RAVV GPΔMuc alone or in the presence of competitor antibodies was assessed by detection with HRP-conjugated anti-biotin antibody.

As shown in FIG. 7A-B, roughly a third of the antibodies in the panel (nine lineages) fell within the RBR antigenic competition group in that they blocked between 64% to greater than 99% of MR191 binding to RAVV GPΔMuc. Two potent neutralizing antibodies, CM1.1 and CM2.1, and a subset of their lineage mates, also fell within this MR191 competition group (FIG. 7A-B). Indeed, antibody CM1.1 which was also used as an antigenic benchmark mAb itself, was the most effective MR191 competitor of all the antibodies tested, knocking out more than 99% of MR191 binding when pre-incubated with RAVV GPΔMuc, better than MR191's competition against itself (FIG. 7A-B). Remarkably, out of the 10 variants that effectively competed more than 65% of MR191's binding to GP, only four effectively competed with benchmark antibody CM1.1, namely, CM2.2, CM3, CM4, and CM2.1 (FIG. 7A-B). The remaining five MR191 competitors, CM5, CM6, CM7, CM8, and CM9, competed to a lesser degree or not at all with CM1.1, suggesting that CM1.1 binding to GP was more difficult to block than MR191's or that the MR191 epitope coincided more directly with these five antibodies. While antibodies CM1.2 and CM2.3 were not effective at competing with either MR191 or CM1.1, these two variants, as noted above, exhibited signs of biochemical instability and were weak binders to GP (FIG. 7A-B; FIG. 4B).

For antigenic benchmark antibody CM10, whose epitope mapped to the GP2 wing (FIG. 6), the assay revealed as expected that pre-incubation of GP with antibodies CM11.1 and CM12.1 reduced CM10 recognition of GP by 94% and 91%, respectively (FIG. 7A-B). Antibodies CM11.1 and CM12.1, like CM10, bound denatured GP by Western blot analysis and their epitopes mapped to the same overlapping residues within the GP2 N terminus as CM10's (FIG. 6). The binding competition assays thus confirmed that all three antibodies, CM10, CM11.1, and CM12.1 fell within the same binding competition group and recognized a common overlapping GP2 wing epitope in the context of the GPΔMuc ectodomain (FIG. 7A-B; FIG. 7E). Two lineage mates of CM11.1 and CM12.1, CM11.2 and CM12.2, respectively, were not evaluated in these assays but were confirmed in a parallel study to target the same GP2 epitope (B. Janus, G. Ofek, unpublished results). None of the other antibodies in the panel successfully competed with CM10 for GP recognition (FIG. 7A-B).

For antigenic benchmark antibody CM13, whose epitope mapped to a contiguous region on GP1 in the vicinity of the RBR, the binding competition assays revealed that three other antibodies fell within its antigenic competition group: CM14, CM15, and CM16. Pre-incubation of RAVV GPΔMuc with any one of these three antibodies blocked CM13 recognition of GP by 40%-52% (FIG. 7A-B). None of these mAb competitors recognized denatured GP by Western-blot analysis, suggesting their epitopes were conformational in contrast to that of CM13. It was noted that antibody CM21, could not be map by pepscan analysis but appears to recognize denatured GP1 by Western-blot analysis, blocked CM13 binding to GP by ˜25%, indicating possible overlap in their epitopes (FIG. 6, FIGS. 7A-B and 7E).

The binding competition assays also revealed that pre-incubation of RAVV GPΔMuc with several antibodies in the panel could enhance benchmark antibody binding to GP. In particular, mAbs CM1.1 and CM2.2, within the RBR-directed antigenic competition group, enhanced the binding of benchmark mAb CM13 to RAVV GPΔMuc by ˜20% (FIG. 7A-B). Since antibody cooperativity in virus neutralization has been reported for orthoebolaviruses, further studies will be necessary to assess whether cooperativity in binding observed here also translates into cooperativity in virus neutralization (56, 57).

The seven remaining GP-reactive antibodies in the panel, CM17 through CM23 did not robustly fall into any of the four antigenic competition groups tested (FIG. 7A-B). These antibodies may target epitopes on RAVV GPΔMuc distinct from those of the benchmark antibodies, although the possibility that the absence of effective competition is a result of insufficient binding affinity to RAVV GPΔMuc as opposed to complementary recognition could not be excluded.

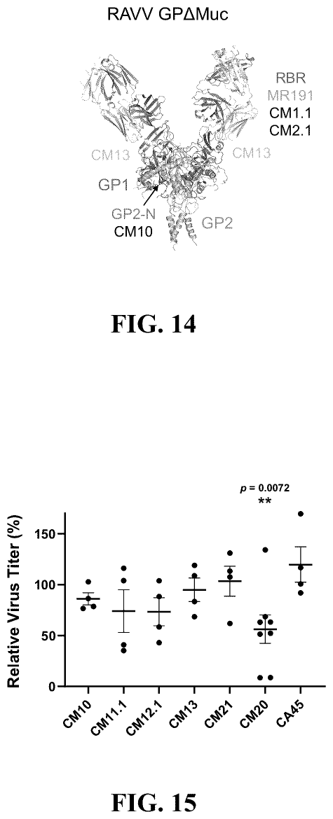

To confirm the GP binding targets of the two antibody lineages that exhibited the highest potency of virus neutralization, CM1 and CM2, their recognition of RAVV GPΔMuc by negative stain electron microscopy (NSEM) was analyzed. Toward this end, fragments of antigen binding (Fabs) of CM1.1 and CM2.1 were expressed and individually complexed with recombinant RAVV GPΔMuc protein. Each complex was applied to EM grids and stained with uranyl formate prior to imaging on a Talos Arctica (200 kV) system. Data processing and 2D classification were performed using RELION (49). As shown in FIG. 7C-D, 2D classes generated for the complexes of CM1.1 Fab and CM2.1 Fab with RAVV GPΔMuc yielded particles with either one or two Fabs bound at the apex of GP. Observed structures were consistent with those observed for antibodies that target the predicted orthomarburgvirus GP RBR, specifically antibodies MR191 and MR78 (FIG. 7E; FIG. 14) (11, 58).

Previous reports indicate that some antibodies that target the mucin-like domain on MARV GP can inhibit virus release from host cells by leading to aggregation of viral particles on the host cell surface (15). Although none of the antibodies in the panel mapped to the mucin-like domain on GP, nonetheless it was sought to assess whether selected antibodies could inhibit viral particle release. Toward this end, an assay that measured the effect of antibodies on MARV-VSV pseudovirus titers released into cell culture supernatants when produced in the presence of individual antibodies was employed (FIG. 15). A non-RBR, non-neutralizing antibodies that bound tightly to GP for testing in this assay was chosen to avoid conflation with inhibition of MARV-VSV entry. Antibodies tested included representatives of all three GP2-wing directed mAb lineages (CM10, CM11.1, and CM12.1), mAb CM13, and mAbs CM20 and CM21 that did not fall into any binding competition group. Orthoebolavirus GP-specific mAb CA45 was assessed in parallel as a negative control. As shown in FIG. 15, the effects of the antibodies tested in this assay varied. When analyzed using an unpaired two-tailed t-test, mAb CM20 exhibited a statistically significant inhibition of MARV-VSV release relative to the no-antibody controls, with a P-value of 0.0072 (FIG. 15). The effects of the other mAbs on MARV-VSV release were not statistically significant. Further studies will be necessary to assess the mechanisms underlying these results and to confirm whether similar results hold when tested against more native filoviral particles.

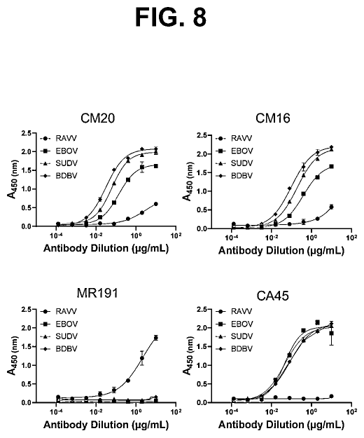

Since animal NHP1 received multivalent immunizations that included SUDV- and EBOV-based antigens, it was next assessed whether any of the 28 antibody lineages could recognize orthoebolavirus GPs as well. Toward that end, all mAbs in the panel were tested for recognition of recombinant EBOV GPΔMuc by ELISA. While a majority of the mAbs had weak to undetectable binding (not shown), two of them—CM16 and CM20—did exhibit measurable binding (FIG. 8). Subsequent assessment of CM16 and CM20 for recognition of other orthoebolavirus GPs, namely SUDV and BDBV GPΔMuc, revealed that both antibodies recognized SUDV and BDBV GPΔMuc equally well if not better than their recognition of EBOV GPΔMuc (FIG. 8). CM16 and CM20 binding to orthoebolavirus GPs was similar to that observed for the control antibody CA45, although their recognition of RAVV GPΔMuc trailed that of the MR191 control (FIG. 8).

Despite only weak or undetectable neutralization of MARV GP pseudoviruses by mAbs CM16 and CM20, in view of their cross-filovirus GP recognition, their neutralization of orthoebolavirus psuedoviruses was measured. Neither CM16 nor CM20 exhibited detectable neutralization of EBOV, SUDV, or BDBV pseudoviruses, suggesting that they either targeted a conserved epitope that does not confer inhibition of entry or that other features rendered them ineffective in preventing viral entry at the concentrations used in these assays (not shown). Taken together, the results confirmed that the multivalent prime-boost immunization regimen given to NHP1 led to the successful induction of monoclonal antibodies with cross-filovirus reactive breadth.

In the present study, a multivalent filovirus prime-boost immunization approach in nonhuman primates to induce immunological breadth against filovirus glycoproteins for downstream mAb isolation was explored. All animals were primed exclusively with MARV GP-based antigens to ensure responses against orthomarburgviruses would effectively take hold in the absence of exposure to GP antigens of other filoviruses. Subsequent repetitive boosting with MARV immunogens alongside SUDV and then EBOV immunogens was not only aimed to induce autologous responses against all three species but also to induce cross-reactive heterologous antibody responses against conserved regions on GP both within and across the Orthoebolavirus and Orthomarburgvirus genera. Indeed, all animals in the study successfully developed both autologous and heterologous antibody titers against multiple filovirus species. Using PBMCs from the animal that exhibited the highest titers of serum antibody responses against Marburg virus GP, a novel panel of cross-reactive GP-specific mAbs were isolated and characterized.

The analysis revealed that roughly a third of the antibodies in the panel mapped to the RBR on GP1, including two lineages—CM1 and CM2—that exhibited potent MARV pseudovirus neutralization. Other than the panel of antibodies isolated from a human survivor of MARV infection and bioinformatically identified homologs thereof, it is believed these antibodies are the only other cases of RBR-directed orthomarburgvirus neutralizing antibodies that have been reported to date, and represent the first such nAbs induced and isolated from animal immunizations (11, 16).

In addition to the RBR binding competition group, three antibody lineages in the panel, CM10, CM11, and CM12, mapped to the GP2 wing protective region (17, 18). CM10, CM11, and CM12 all recognized the same continuous epitope within this region, spanning residues 450-464, that partially or fully overlapped with epitopes of protective mAbs 30G4 and MR228 (17, 18). GP2 residues 450-464 are fully conserved across all MARV isolates but differ within RAVV GP at 5 of 15 residue positions. Since CM10, CM11, and CM12 were all solely induced by MARV-based GP antigens, their cross-reactive recognition of RAVV GP likely relies on conserved residue positions within this region or on accommodation of sequence variation.

The third antigenic competition group identified mapped to an epitope on GP1 that spanned residues 61-75, a partially conserved region across filoviruses. This region was previously identified as the target of a pan-filovirus reactive murine antibody m21D10 (29). Four antibodies in the panel fell within this antigenic group, CM13, CM14, CM16, and CM16. While mAb CM13 bound a peptide spanning this region and to denatured GP, mAbs CM14, CM15, and CM16 did not bind this peptide nor did they recognize denatured GP, suggesting they target conformational or complex epitopes that overlap but are nonetheless distinct from that of CM13.

An additional goal of the present study was to use multivalent prime-boost immunization to induce mAbs with pan-filovirus reactivity. Two mAbs in the panel, CM16 and CM20, were found to possess pan-filovirus reactivity and recognized orthomarburgvirus as well as multiple orthoebolavirus GPs, including SUDV, EBOV, and BDBV. Remarkably, mAb CM16 fell within the CM13 binding competition group, whose epitope on GP1 overlapped that of pan-reactive murine antibody m21D10 (29), consistent with definition this region on GP1 as a pan-reactive target.

Lastly, it was noted that seven antibodies in the panel, including pan-filovirus reactive mAb CM20, could not be unambiguously mapped to any known site on GP, posing the possibility of additional antigenic targets on GP that have yet to be fully defined. Taken together, the disclosed Example above expands the available repertoire of mAbs directed against orthomarburgvirus GP, including novel neutralizing lineages targeting the RBR. It also provides an understanding of orthomarburgvirus GP antigenicity and determinants of antibody cross-reactivity.

TABLE I
Heavy Chain CDR Sequences

| | CDRH1 | CDRH2 | CDRH3 |
|---|---|---|---|
| CM25 | GGSISGGYG | IYSSSGNT | ARTPVLLLLGDY |
| CM1.1 | GGSISSNY | IYGTGSTT | ARETPVLHFVEWFGSLDV |
| CM1.2 | GGSISSNY | IYGSGTTT | ARETPVLHFLEWFGSLDV |
| CM28 | GYTFTRYY | INPSNGNT | ARNIDS |
| CM26 | GASTSSSW | INGKTGST | ARVYAGEAGY |
| CM11.2 | GFTFGDYG | ISSGSSYI | TRGRVYSGYREFDY |
| CM7 | GFTFSSYG | IWSDGIKT | ARNEYNFWSGYTYYGLDS |
| CM8 | GGSISDSYY | IYGSGGST | ARGFTIFGLVIYNRFDV |
| CM21 | GFTFSSYD | ISYTGKTI | TGWNKVTLDV |
| CM2.1 | GFTFSNYY | INTGGGYT | AKDRQVLQFLDWLTYGLDS |
| CM2.3 | GFTFSSYY | INTGGGST | AKDRQVLQFLDWLTYGLDS |
| CM2.2 | GFTFSNYY | INTAGGYT | AKDRQVLQFLDWLTYGLDS |
| CM24 | GFTFSDHY | IRNQANGGTA | VRGGTGGGRY |
| CM12.1 | GFTFSSYG | INSGGGST | AKFGWAVPGLDY |
| CM4 | GGSISDSYR | IYGSATST | ASEYYYSGTYYGYYGLDS |
| CM5 | GFTFSDYY | RNKANGGTA | ARVSYVLQFLEWLTLDY |
| CM23 | GGSISDSYR | IYGSSTST | ARDGGGFTIFGVVMVFDY |
| CM11.1 | GFTFGDYG | ISSGSGYI | TRGREYSGYREFDF |
| CM20 | GGSFSGYY | ISGSSGNT | ARVGYNFWTGPDV |
| CM17 | GFTFSSYA | INSGGGST | AKDLLGGSSWSNSLDV |
| CM13 | GFTFGDYG | ISSDSSYI | TRDRGSRFDV |
| CM9 | GFTFSNYD | VSYTGKTI | TRGNPPVQFLEWLPIDY |
| CM6 | GGSISDSYY | IYGSGGST | AKDNRLTIFGLTFIDY |
| CM10 | GFTFGDYG | ISSGSGYI | SRGGIPAGPNRYGLDS |
| CM12.2 | GFTFSTYG | ISSGGVT | AKFGWAVPGLDY |
| CM18 | GGSFSGYY | ISGSSGST | ARLGVSGSWFWFRFDV |
| CM14 | GFTFSSYG | ISSASSYI | TSGGSGSGWSTLSFDY |
| CM22 | GYSFTVSW | IYPGDSDT | AKARWSIFYGLDS |
| CM19 | GGSISDSYY | IYGSGGST | ARDPLLQFLDWLFMDV |
| CM27 | GFTFSDYA | IRSKYNNYAT | TTDHDTTIFGVVIPYYGLDS |
| CM16 | GYTFTDYY | INPKTGGT | ESSRIVGTKYFEF |
| CM3 | GGSISDSYR | IYGSSTTT | ASPPVLQFLEWTHFEV |
| CM15 | GFIFSDYY | IRNKAKGGTA | VFEHYSSNYRVDY |

TABLE II
Light Chain CDR Sequences

| | CDRL1 | CDRL2 | CDRL3 |
|---|---|---|---|
| CM25 | GGINVAGYH | YKSDSDK | ATGHSSGWV |
| CM1.1 | SSNIGADYY | ENN | LAWDNSLNTAL |
| CM1.2 | SSNIGADYY | ENN | LAWDNSLNTAL |
| CM28 | GGINVAGYH | YKSDSDK | AIGHSSGPV |
| CM26 | QSISSW | KGS | LQYSSSPFT |
| CM11.2 | SSNIGGYY | ENN | QSYDSSLSAWV |
| CM7 | NIGSEA | SDR | QVYDISGIMS |
| CM8 | QSVSSN | YAS | QQESNWPLT |
| CM21 | SSIIGGYY | EKN | QSYDSSLSALV |
| CM2.1 | SSNFGRNY | NNN | SAWDNSLSGL |
| CM2.3 | SSNIGSNY | NNN | SAWDNSLSGL |
| CM2.2 | SSNIGSNS | NNN | SAWDNSLRGL |
| CM24 | QSLVYSDGKTY | QVS | MQGTHWPLT |
| CM12.1 | QSLVHSDGKTY | QVS | MQGTHWPFT |
| CM4 | KIGSKY | YDS | QVWDSSSDHYI |
| CM5 | SGISVGGYN | YYSDSNK | TTWHNNAWV |
| CM23 | QGISSY | YAN | QQGNSNPYS |
| CM11.1 | SSNIGGYY | ENN | QSYDSSLSAWV |
| CM20 | SSDIGGYNY | EVS | SSYAGSYTFL |
| CM17 | SSNIGAGYY | ENN | LAWDNSLSAGL |
| CM13 | QSLLDSEDGNTY | EVS | MQALDFPPT |
| CM9 | QSVSSF | GAS | YQHSSGYT |
| CM6 | SDLSVGSKT | YYSDSDK | QVYDSSAGL |
| CM10 | QSLLHSGGKTY | EVS | MQGIQLPWT |
| CM12.2 | QSLVHSDGKTY | QVS | MQGTHWPFT |
| CM18 | QGISDY | AAS | LQGYSTPYSFG |
| CM14 | QSISSW | KAS | LQYSSSPFTFG |
| CM22 | SSNIGGYY | QDN | GAWDSSLSAYI |
| CM19 | QGISSY | AAS | QQHNSYPRT |
| CM27 | SDYSNYA | VGSGGIVG | GADHGTGSSFVLG |
| CM16 | QSLLHSNGY | LGS | LQDIQLPLT |
| CM3 | QGISSY | YTN | QQGNSYPYS |
| CM15 | SSNIGPYY | DNN | SAWDSSLSAVL |

TABLE III
>RAVN GP dMUC WT (Foldon, TEV, Strep, Linker, 8xHis, Avitag) (EcoRI, Kozak,
BamHI, HindIII)
gaattcccaccMKTIYFLISLILIQSIKTLPVLEIASNSQPQDVDSVCSGTLQKTEDVHLMGFTLSGQKVADSPLEA
SKRWAFRTGVPPKNVEYTEGEEAKTCYNISVTDPSGKSLLLDPPSNIRDYPKCKTVHHIQGQNPHAQGIALHLWGAF
FLYDRVASTTMYRGKVFTEGNIAAMIVNKTVHRMIFSRQGQGYRHMNLTSTNKYWTSSNETQRNDTGCFGILQEYNS
TNNQTCPPSLKPPSLPTVTPSIHSTNTQINTAKSGTRPPIYFRKKRSILAKEGDIGPNLDGLINTEIDFDPIPNTET
IFDESPSFNTSTNEEQHTPPNISLTFSYFPDKNGDTAYSGENENDCDAELRIWSVQEDDLAAGLSWIPFFGPGIEGL
YTAGLIKNQNNLVCRLRRLANQTAKSLELLLRVTTEERTFSLINRHAIDFLLTRWGGTCKVLGPDCCIGIEDLSKNI
SEQIDKIRKDEQKEETGSGYIPEAPRDGQAYVRKDGEWVLLSTFLGTENLYFQSSAWSHPQFEKGGGSGGGSGGSAW
SHPQFEKHHHHHHHHSGLNDIFEAQKIEWHEtgaaagctt
Amino Acid Sequences
>CM25 heavy chain
QVQLQESGPGLLKPSETLSLTCAVSGGSISGGYGWGWIRQPPGKGLEWIGSIYSSSGNTY
YNPSLKSRVIISSDTSKNQFSLKLRSVTAADTAVYYCARTPVLLLLGDYWGQGVLVTVSS
>CM1.1 heavy chain
QVQLQESGPGLVKPSETLSLTCAVSGGSISSNYWSWIRQAPGKGLEWIGYIYGTGSTTNY
NPSLKSRVTLSVDTSKKQFSLSLSSVTAADTAVYYCARETPVLHFVEWFGSLDVWGRGFL
VTVSS
>CM1.2 heavy chain
QVQLQESGPGLVKPSETLSLTCAVSGGSISSNYWSWIRQAPGKGLEWIGYIYGSGTTTNY
NPSLKSRVTLSVDTSKNQFSLKLSSVTAADTAVYYCARETPVLHFLEWFGSLDVWGRGVL
VTVSS
>CM28 heavy chain
QVQLVQSGGEVKKPGTSVKLSCKDFGYTFTRYYIWVRQAPGQVLEWMGWINPSNGNTAYA
QEFQGRVTMTRDTSTNTAYREGNSLRYEDKAVYYCARNIDSWGQGVLVTVSS
>CM26 heavy chain
QVQLQESGPGLVKPSETLSLTCTVSGASTSSSWWSWIRQSPGKGLEWIGEINGKTGSTNY
NPSLKSRVTISKDASKNQFSLKLTSVTAADTAVYYCARVYAGEAGYWGQGVLVTVSS
>CM11.2 heavy chain
EVQLVESGGGLVQPGGSLRLSCAVSGFTFGDYGMHWVRQAPGKGLEWVSAISSGSSYIYY
ADSVKGRFTISRDNAKNSLSLQMSSLRAEDTAVYYCTRGRVYSGYREFDYWGQGVLVTVS
S
>CM7 heavy chain
EVQLVESGGGLVQPGGSLRLSCAASGFTFSSYGMHWVRQAPGKGLEWVAFIWSDGIKTYY
ADSVMDRFTISRDNSKNMLYLQMNNLKLEDTAVYYCARNEYNEWSGYTYYGLDSWGQGVV
VTVSS
>CM8 heavy chain
QVQLQESGPGLVKPSETLSLTCAVSGGSISDSYYWSWIRQSPGKGLEWIGYIYGSGGSTY
YNPSLKSRVTISTDTSKNQFSLKLSSVTAADTAVYYCARGFTIFGLVIYNRFDVWGPGVL
VTVSS
>CM21 heavy chain
EVQLVESGGGLVQPGGSLRLSCAASGFTFSSYDMSWVRQAPGKGLEWVSYISYTGKTIYY
ADSVKGRFTISRDNAKNSLSLQMSSLRAEDTAVYYCTGWNKVTLDVWGRGLLVTVSS
>CM2.1 heavy chain
EVQLVESGGGLAKPGGSLRLSCAASGFTFSNYYMYWVRQAPGKGLEWVSAINTGGGYTYY
ADSVKGRFTISRDNSKNTLSLQMNSLRAEDTAVYYCAKDRQVLQFLDWLTYGLDSWGQGV
VVTVSS
>CM2.3 heavy chain
EVQLVESGGGLAKPGGSLRLSCAASGFTFSSYYMYWVRQAPGKGLEWVSAINTGGGSTYY
ADSVKGRFTISRDNSKNTLSLQMNSLRAEDTAVYYCAKDRQVLQFLDWLTYGLDSWGQGV
VVTVSS
>CM2.2 heavy chain
EVQLVESGGGLAKPGGSLRLSCAASGFTFSNYYMYWVRQAPGKGLEWVSAINTAGGYTYY
ADSVKGRFTISRDNSKNTLSLQMNSLRAEDTAVYYCAKDRQVLQFLDWLTYGLDSWGQGV
VVTVS
>CM24 heavy chain
EVQLVESGGGLVQPGGSLRLSCAASGFTFSDHYMSWVRQAPGKGPEWIGFIRNQANGGTA
EYAASVKGRFTISRDDSKTIASLQMNSLNTEDTAVYYCVRGGTGGGRYWGQGVLVTVSS
>CM12.1 heavy chain
EVQLVETGGGLVQPGGSLKLSCAASGFTFSSYGMSWVRQAPGKGLEWVSSINSGGGSTHY
ADSVKGRFTISRDNSKNTLSLQMNSLRAEDTAVYYCAKFGWAVPGLDYWGQGVLVTVSS
>CM4 heavy chain
QVQLQESGPGVVKPSETLSLTCAVSGGSISDSYRWSWIRQPPGKGLAWIGYIYGSATSTN
YNPSLKSRVTISKDTSQNQFSLKLSSVTAADTAVYYCASEYYYSGTYYGYYGLDSWGQGV
VVTVSS
>CM5 heavy chain
EVQLVESGGGLVQPGGSLRLSCAASGFTFSDYYMQWVRQAPGKGPEWVGFIRNKANGGTA
EYAASVKGRFTISRDDSKSIASLQMNSLKTEDTAVYYCARVSYVLQFLEWLTLDYWGQGV
LVTVSS
>CM23 heavy chain
QVQLQESGPGVVKPSETLSLTCAVSGGSISDSYRWSWIRQPPGKGLEWIGYIYGSSTSTN
YNPSLKSRVTISKDTSKNQFSLKLSSVTAADTAVYYCARDGGGFTIFGVVMVFDYWGQGV
LVTVSS
>CM11.1 heavy chain
EVQLVESGGGLVQPGGSLRLSCAASGFTFGDYGMHWVRQAPGKGLEWVSAISSGSGYIYY
ADSVKGRFTISRDNAKNSLSLQMSSLRAEDTAVYYCTRGREYSGYREFDFWGQGVLVTVS
S
>CM20 heavy chain
QVQLQESGPGLVKPSETLSLTCTVSGGSFSGYYWGWIRQPPGKGLEWIAYISGSSGNTDY
NPSLKSRVTISTDTSKNQFSLKLSSVTAADTAVYYCARVGYNFWTGPDVWGRGVLVTVSS
>CM17 heavy chain
EVQLVETGGGLVQPGGSLRLSCAASGFTFSSYAMQWVRQAPGKGLEWISAINSGGGSTYY
ADSVKGRFTISRDNSKNTLSLQMNSLRAEDTAVYYCAKDLLGGSSWSNSLDVWGRGVLVT
VSS
>CM13 heavy chain
EVQMVESGGGLVQPGGSLRLSCAASGFTFGDYGMHWVRQAPGKGLEWVSSISSDSSYIYY
PDSVKGRFTISRDNAKNSLSLQMRSLGAEDTAVYYCTRDRGSRFDVWGPGVLVTVSS
>CM9 heavy chain
EVQLVESGGGLVQPGGSLRLSCAASGFTFSNYDMSWVRQAPGKGLEWVSYVSYTGKTIYY
ADSVKGRFTISRDNAKKSLSLQMSSLRAEDTAVYYCTRGNPPVQFLEWLPIDYWGQGVLA
TVSSC
>CM6 heavy chain
QVQLQESGPRLVKPSETLSLTCAVSGGSISDSYYWSWIRQSPGKGLEWIGYIYGSGGSTY
YNPSLKSRVTISTDTSKNQFSLKLSSVTAADTAVYYCAKDNRLTIFGLTFIDYWGQGVLV
TVSS
>CM10 heavy chain
EVQLVESGGGLVRPGGSLRLSCAASGFTFGDYGMHWVRQAPGKGLEWVSTISSGSGYIYY
ADSVKGRFTISRDNAKNSLSLQMSSLRVEDTAVYYCSRGGIPAGPNRYGLDSWGQGVVVT
VSS
>CM12.2 heavy chain
EVQLVETGGGLVQPGGSLKVSCAASGFTFSTYGMSWVRQAPGKGLEWVSSISSGGVTHYA
DSVKGRFTISRDDSKNTLSLQMNSLKTEDTAVYYCAKFGWAVPGLDYWGQGVLVTVSS
>CM18 heavy chain
QVQLQESGPGLVKPSETLSLTCAVSGGSFSGYYWGWIRQPPGKGLEWIGYISGSSGSTDY
NPSLKSRVTISTDTSKNQFSLKLNSVTAADTAVYYCARLGVSGSWFWFRFDVWGPGVLVT
VSS
>CM14 heavy chain
EVQLVESGGGLVQPGGSLRLSCAASGFTFSSYGMSWVRQAPGKGLEWVSSISSASSYIYY
ADSVKGRFTISRDNAKNSLSLQMNSLRAEDTAVYYCTSGGSGSGWSTLSFDYWGQGVLVT
VSS
>CM22 heavy chain
EVQLVQSGAEVKRPGESLRISCKTSGYSFTVSWISWVRQMPGKGLEWMGSIYPGDSDTRY
HPSFQGHVTISADKSISTTYLQWSSLKASDTATYYCAKARWSIFYGLDSWGQGVVVTVSS
>CM19 heavy chain
QVQLQESGPGLVKPSETLSLTCAVSGGSISDSYYWSWIRQSPGKGLEWIGYIYGSGGSTY
YNPSLKSRVTISTDTSKNQFSLKLSSVTAADTAVYYCARDPLLQFLDWLFMDVWGRGVLV
TVSS
>CM27 heavy chain
EVQLVESGGDLVQPGGSLRLSCAASGFTFSDYAMSWVRQASGKGLEWVGSIRSKYNNYAT
EYAASVKGRFTISRDDSKNTLYLQMNSLKTEDTAVYYCTTDHDTTIFGVVIPYYGLDSWG
QGVVVTVSS
>CM16 heavy chain
QVQLVQSGAEVKKPGSSVKVSCKASGYTFTDYYMHWVRQAPGQGLEWMGEINPKTGGTNY
AQKFQGRVTMTRDTSTSTAYMELSSLRSEDTAVYYCESSRIVGTKYFEFWGQGALVTVSS
>CM3 heavy chain
QVQLQESGPGMVKPSETLSLTCAVSGGSISDSYRWTWIRQPPGKGLEWIGYIYGSSTTTN
YNPSLKSRVTISKDTSKNQFSLNLRSVTVADTALYYCASPPVLQFLEWTHFEVWGQGALV
TVSS
>CM15 heavy chain
EVQLVESGGGLVQPGGSLRLSCAASGFIFSDYYMSWVRQVPGKGPEWVGFIRNKAKGGTA
EYAASVKGRFTISRDDSKSIASLQMNSLNTEDTAVYYCVFEHYSSNYRVDYWGQGVLVTV
SS
>CM25 light chain
QSVLTQPASLSASPGASASLTCTFSGGINVAGYHILWYQQKPGSPPRYLLRYKSDSDK~G
QGSGVPSRFSGSKDASANTGILRISGLQSEDEADYYCATGHSSGWVFGGGTRLT
>CM1.1 light chain
QSVLTQPPSASGAPGQRVTISCTGSSSNIGADYYVSWYQQFPGTAPKLLIYENNKRPSGV
SDRFSGSKSGTSASLTITGLQSEDEADYYCLAWDNSLNTALFGGGTRLT
>CM1.2 light chain
QSVLTQPPSASGAPGQRVTISCTGSSSNIGADYYVSWYQQFPGTAPKLLIYENNKRPSGV
SDRFSGSKSGTSASLTITGLQSEDETDYYCLAWDNSLNTALFGGGTRLT
>CM28 light chain
QSVLTQPASLSASPGASASLTCTFSGGINVAGYHIFWFQQKPGSPPRYLLRYKSDSDKGQ
GSGVPSRFSGSKDASANTGILRICGLQCEDEAEYYCAIGHSSGPVFGGGTRLT
>CM26 light chain
DIQMTQSPSSLSASVGDTVTITCRASQSISSWLDWYQQKPGKAPKVLIYKGSSLESGVPS
RFSGSGSGTDFTLTISSLQPEDFATYYCLQYSSSPFTFGPGTKLDIK
>CM11.2 light chain
QSVLTQPPSVSGAPGQRVTISCTGSSSNIGGYYVQWYQQLPGTAPKLLIYENNKRPSGVS
DRESGSQSGTSASLTITRLQSEDEADYYCQSYDSSLSAWVLGGGTRLT
>CM7 light chain
SYELTQPPSVSVSPGQTARITCGGDNIGSEAVHWYQQKPPQAPVQVIYSDRERPSGIPER
FSGSKSGNTATLTISGVEAGDEADYYCQVYDISGIMSTLFGGGTRLT
>CM8 light chain
EIVMTQSPASLSLSPGERATLSCRASQSVSSNLAWYQQKPGQAPRLLIYYASNRATGIPD
RFSGSGSGTDFTLTISSLEPEDVGVYYCQQESNWPLTFGGGTKVELK
>CM21 light chain
QSVLTQPPSVSGAPGQRVTISCTGSSSIIGGYYVQWYQQLPGTAPKLLIYEKNKRPSGVS
DRFSGSQSGTSASLTITGLQSEDEADYYCQSYDSSLSALVEGSGTRLT
>CM2.1 light chain
QSVLTQPPSASGAPGQSVTISCSGSSSNFGRNYVYWYQHLSGKAPKLLIYNNNQRPSGVP
DRFSGSKSGTSASLAISGLQSEDEADYYCSAWDNSLSGLFGGGTRLT
>CM2.3 light chain
QSVLTQPPSASGAPGQSVTISCSGSSSNIGSNYVYWYQQLSGKAPKLLIYNNNQRPSGVP
DRFSGSKSGTSASLAISGLQSEDEADYYCSAWDNSLSGLFGGGTRLT
>CM2.2 light chain
QSVLTQPPSASGAPGQTVTISCSGSSSNIGSNSVYWYQQLSGKAPKLLIYNNNQRPSGVP
DRFSGSKSGTSASLAISGLQSEDEADYYCSAWDNSLRGLFGGGTRLT
>CM24 light chain
DVVMTQSPLSLPVTPGQPASISCRSSQSLVYSDGKTYLNWLQQKSGQPPRRLIYQVSNRD
SGVPDRFSGSGTGTDFTLKISRVEAEDVGVYYCMQGTHWPLTFGGGTMVELK
>CM12.1 light chain
DVVMTQSPVSLPVTLGQPASISCRSSQSLVHSDGKTYLNWLQQKPGQPPRRLIYQVSNRD
SGVPDRFSGSGAGTDFTLKISRVEAEDVGVYYCMQGTHWPFTFGPGTKLDIK
>CM4 light chain
QSVLTQPPSVSVSPGQTARITCGGDKIGSKYVHWYQQKPPQAPVLVIYYDSERPSGIPER
FSGSKSGNTATLTISGVEAGDEADYYCQVWDSSSDHYIFGAGTRLT
>CM5 light chain
QSVLTQPTSLSASPGASVRLTCTLRSGISVGGYNIHWYQQKPGSPPRYLLYYYSDSNKAQ
GSGVPSRFSGSKDASANAGILLISGFQSEDEADYYCTTWHNNAWVEGGGTRLT
>CM23 light chain
DIQMSQSPSSLSASVGDRVTITCRASQGISSYLNWYQQKPGKAPKLLIYYANSLASGVPS
RFSGSGSGTEFTLTISSLQPEDFATYYCQQGNSNPYSFGQGTKVEIK
>CM11.1 light chain
QSVLTQPPSVSGAPGQRVTISCTGSSSNIGGYYVQWYQQLPGTAPKLLIYENNKRPSGVS
DRFSGSQSATSASLTITGLQSEDEADYYCQSYDSSLSAWVEGGGTRLT
>CM20 light chain
QSVLTQPRSVSGSPGQSVTISCTGTSSDIGGYNYVSWYQQHPGTAPKLMIYEVSKRPSGV
SDRFSGSKSGNTASLTISGLQAEDEADYYCSSYAGSYTFLFGGGTRLT
>CM17 light chain
QSVLTQPPSASGAPGQRVTISCTGSSSNIGAGYYVSWYQQFPGTAPKLLIYENNKRPSGV
SDRFSGSKSGTSASLTITGLQSEDEADYYCLAWDNSLSAGLFGGGTRLT
>CM13 light chain
DIVMTQTPLSLPVTLGEPASISCRSSQSLLDSEDGNTYLEWYLQKPGQSPQLLIYEVSNRASGV
PDRFSGSGSDTDFTLKISRVEAEDVGVYYCMQALDFPPTFGQGTKVEIK
>CM9 light chain
QVILTQSPATLSLSPGERATLSCRASQSVSSFLAWYQQKPGQAPRLLIYGASSRATGIPD
RFSGSGSGTDFTLTISSLEPEDVGVYHCYQHSSGYTFGGGTKVELK
>CM6 light chain
QSVLTQPPSLSASPGASARLPCTLSSDLSVGSKTMYWYQQKPGSAPRLFLYYYSDSDKQL
GPGVPNRVSGSKETSSNTAFLLISGLQPEDEADYYCQVYDSSAGLLFGGGTRLT
>CM10 light chain
DIVMTQTPLSLPVTPGEPASISCRSSQSLLHSGGKTYLYWYLQKPGQSPQLLIYEVSNRA
SGVPDRFTGSGSGTDFTLRISRVEAEDVGVYYCMQGIQLPWTFGQGTKVEIK
>CM12.2 light chain
DVVMTQSPVSLPVTLGQPASISCRSSQSLVHSDGKTYLNWLQQKPGQPPRRLIYQVSNRD
SGVPDRFSGSGAGTDFTLKISRVEAEDVGVYYCMQGTHWPFTFGPGTKLDIK
>CM18 light chain
DIQMSQSPSSLSASVGDRVTITCRASQGISDYLNWYQQKPGKAPNLLIYAASSLQSGVPS
RFSGSGSGTDFTLTISSLQPEDFAAYYCLQGYSTPYSFGQGTKVEIK
>CM14 light chain
DIQMTQSPSSLSASVGDTVTITCRASQSISSWLAWYQQKPGKAPKLLIYKASSLQSGVPS
RFSGSGSGTDFTLTISSLQPEDFAAYYCLQYSSSPFTFGPGTKLDIK
>CM22 light chain
QSVLTQPPSVSGDPGQRVTISCTGSSSNIGGYYVYWYQQFPGTAPKLLIYQDNKRPSGVS
DRFSGSKSGTSASLTITGLQPGDEADYYCGAWDSSLSAYIFGAGTRLT
>CM19 light chain
DIQMTQSPSSLSASVGDRVTITCRASQGISSYLAWYQQKPGKAPKLLIYAASTLQSGVPS
RFSGSGSGTDFTLTISSLQPEDFATYYCQQHNSYPRTFGQGTKVEIK
>CM27 light chain
QSVLTQPPSASASLGASVTLTCTLSDYSNYAVDWHPQRPGKGPQFVMRVGSGGIVGSKGD
GIPDRFSGSGSGLIRYLTIKNIQEEDESDYHCGADHGTGSSFVLGIRRRDPADTRLT
>CM16 light chain
DIVMTQTPLSLPVTPGEPASISCRSSQSLLHSNGYTYLFWYLQKPGQSPQLLIYLGSNRA
SGVPDRFSGSGSGTDFTLKISRVEAEDVGVYYCLQDIQLPLTFGGGTKVELK
>CM3 light chain
DIQMSQSPSSLSASVGDRVTITCRASQGISSYLNWYQQKPGKAPNLLIYYTNNLASGVPS
RFSGSGSGTEFTLTISSLQPEDFATYYCQQGNSYPYSFGQGTNLEIK
>CM15 light chain
QSVLTQPPSVSGDPGQRVTISCTGSSSNIGPYYVYWYQQFPGTAPKLLIYDNNKRPSGVS
DRFSGSKSGTSASLTITGLQPGDEADYYCSAWDSSLSAVLFGGGTRLT
Nucleotide Sequences Native
>CM25 heavy chain
CAGGTGCAGCTGCAGGAGTCGGGCCCAGGACTGTTAAAGCCTTCAGAGACCCTGTCCCTC
ACCTGCGCTGTCTCTGGTGGCTCCATCAGCGGTGGTTATGGCTGGGGCTGGATCCGCCAG
CCCCCAGGGAAGGGGCTGGAGTGGATTGGGAGTATCTATAGTAGTAGTGGGAACACCTAC
TACAACCCCTCCCTCAAGAGTCGAGTCATCATTTCATCAGACACGTCCAAGAACCAGTTC
TCCCTGAAGCTGAGGTCTGTGACCGCCGCGGACACGGCCGTGTATTACTGTGCGAGAACT
CCCGTGCTACTACTACTTGGAGACTACTGGGGCCAGGGAGTCCTGGTCACCGTCTCCTCA
>CM1.1 heavy chain
CAGGTGCAGCTACAGGAGTCGGGCCCAGGACTGGTGAAGCCTTCGGAGACCCTGTCCCTC
ACCTGCGCTGTCTCTGGTGGCTCCATCAGCAGTAACTACTGGAGCTGGATCCGCCAGGCC
CCAGGGAAGGGACTGGAGTGGATTGGGTATATCTATGGTACTGGTAGTACCACCAACTAC
AACCCCTCCCTCAAGAGTCGAGTCACCCTGTCAGTGGACACGTCCAAGAAGCAGTTCTCC
CTGAGTCTGAGCTCTGTGACCGCCGCGGACACGGCCGTTTATTATTGTGCGAGAGAAACC
CCCGTATTACATTTTGTGGAGTGGTTTGGTTCATTGGATGTCTGGGGCCGGGGATTTCTG
GTCACCGTCTCCTCA
>CM1.2 heavy chain
CAGGTGCAGCTGCAGGAGTCGGGCCCAGGACTGGTGAAGCCTTCGGAGACCCTGTCCCTC
ACCTGCGCTGTCTCTGGTGGCTCCATCAGCAGTAACTACTGGAGCTGGATCCGCCAGGCC
CCAGGGAAGGGACTGGAGTGGATTGGGTATATCTATGGTAGTGGTACTACCACCAACTAC
AACCCCTCCCTCAAGAGTCGAGTCACCCTGTCAGTAGACACGTCCAAGAACCAGTTCTCC
CTGAAGCTGAGCTCTGTGACCGCCGCGGACACGGCCGTGTATTACTGTGCGAGAGAAACC
CCCGTATTACACTTTTTGGAGTGGTTTGGTTCATTGGATGTCTGGGGCCGGGGAGTTCTG
GTCACCGTCTCCTCA
>CM28 heavy chain
CAGGTCCAGCTGGTGCAATCCGGGGGTGAGGTGAAGAAGCNTGGGACCTCAGTGAAGCTT
TCCTGCAAAGATTTTGGTTACACCTTTACCAGGTANTATATAAANTGGGTGAGACAGGCC
CCTGGACAAGTGCTTGAGTGGATGGGATGGATTAACCCTAGCAATGGTAATACAGCCTAA
GCACAGGAGTTCCAGGGCAGAGTCACCATGACCAGGGACACGTCCANGAATACAGCCTAC
AGGGAGGGGAACAGCCTAAGATATGAGGACAAGGCCGTGTATTACNGTGTAAGAGTCNTT
ATCTNNTGGGGCCAGGGAGTCCTGGTCACCGNNTCCTCT
>CM26 heavy chain
CAGGTGCAGCTGCAGGAGTCGGGCCCAGGACTGGTGAAGCCTTCGGAGACCCTGTCCCTC
ACCTGCACTGTCTCTGGTGCCTCCACTAGTAGTAGCTGGTGGAGCTGGATCCGTCAGTCC
CCAGGGAAGGGACTGGAGTGGATTGGGGAGATCAATGGTAAAACTGGGAGCACTAACTAC
AACCCCTCCCTCAAGAGTCGAGTCACCATTTCAAAAGACGCGTCCAAGAACCAGTTCTCC
CTGAAGCTGACCTCTGTGACCGCCGCGGACACGGCCGTATATTACTGTGCGAGAGTTTAC
GCTGGGGAAGCGGGTTACTGGGGCCAGGGAGTCCTGGTCACCGTCTCCTCA
>CM11.2 heavy chain
GAGGTGCAGCTGGTGGAGTCTGGGGGAGGCTTGGTCCAGCCTGGGGGGTCCCTGAGACTC
TCCTGTGCAGTCTCTGGATTCACCTTTGGTGATTATGGCATGCACTGGGTCCGCCAGGCT
CCGGGAAAGGGGCTGGAGTGGGTCTCAGCCATTAGTAGTGGTAGTAGTTACATATACTAC
GCTGACTCCGTGAAGGGCCGATTCACCATCTCCAGAGACAACGCCAAGAATTCGCTGTCT
CTGCAAATGAGCAGCCTGAGAGCCGAGGACACGGCCGTGTATTACTGTACTAGAGGCCGG
GTATACAGCGGGTACCGAGAATTTGACTACTGGGGCCAGGGAGTCCTGGTCACCGTCTCC
TCA
>CM7 heavy chain
GAGGTGCAGCTGGTGGAGTCTGGAGGAGGCTTGGTTCAGCCTGGGGGGTCCCTGAGACTT
TCCTGTGCAGCCTCTGGATTCACCTTCAGTAGCTATGGCATGCACTGGGTCCGCCAGGCT
CCGGGGAAGGGGCTGGAGTGGGTGGCATTTATATGGTCTGATGGAATTAAGACATACTAC
GCAGACTCTGTGATGGACCGATTCACCATCTCCAGAGACAATTCCAAGAACATGCTATAT
CTTCAAATGAACAACCTGAAATTGGAGGACACGGCCGTGTATTACTGTGCGAGAAACGAA
TATAATTTTTGGAGTGGTTACACGTACTACGGTTTGGATTCCTGGGGCCAAGGGGTCGTC
GTCACCGTCTCCTCA
>CM8 heavy chain
CAGGTGCAGCTGCAGGAGTCGGGCCCAGGACTGGTGAAGCCTTCGGAGACCCTGTCCCTC
ACCTGCGCTGTCTCTGGGGGCTCCATCAGCGATAGTTACTACTGGAGCTGGATCCGCCAG
TCCCCAGGGAAGGGACTGGAGTGGATTGGGTACATCTATGGTAGTGGTGGTAGCACCTAC
TACAACCCCTCCCTCAAGAGTCGAGTCACCATTTCAACAGACACATCCAAGAACCAGTTC
TCCCTGAAGCTGAGCTCTGTGACCGCCGCGGACACGGCCGTGTATTACTGTGCGAGAGGG
TTTACAATTTTTGGACTGGTTATCTACAACCGGTTCGATGTCTGGGGCCCGGGAGTCCTG
GTCACCGTCTCCTCA
>CM21 heavy chain
GAGGTGCAGCTGGTGGAGTCTGGGGGAGGCTTGGTCCAGCCTGGAGGGTCCCTGAGACTC
TCCTGTGCAGCCTCTGGATTCACCTTCAGTAGTTACGACATGAGCTGGGTCCGCCAGGCT
CCGGGAAAGGGGCTGGAGTGGGTCTCATATATTAGTTACACTGGTAAAACCATATACTAC
GCTGACTCCGTGAAGGGCCGATTCACCATCTCCAGAGACAACGCCAAGAACTCGCTGTCT
CTGCAAATGAGCAGCCTGAGAGCCGAGGACACGGCCGTGTATTACTGTACGGGTTGGAAC
AAAGTGACATTGGATGTCTGGGGCCGGGGACTTCTGGTCACCGTCTCCTCA
>CM2.1 heavy chain
GAGGTGCAGCTGGTGGAGTCTGGGGGCGGCTTGGCAAAGCCTGGGGGGTCCCTGAGACTC
TCCTGTGCAGCCTCTGGATTCACCTTCAGTAACTACTACATGTACTGGGTCCGCCAGGCT
CCAGGGAAGGGGCTAGAGTGGGTCTCAGCTATTAATACTGGTGGGGGTTACACATACTAC
GCAGACTCCGTGAAGGGCCGATTCACCATCTCCAGAGACAACTCAAAGAACACGCTCTCC
CTGCAAATGAACAGCCTGAGAGCTGAGGACACGGCCGTGTATTACTGTGCGAAAGATCGG
CAAGTATTACAATTTTTGGACTGGCTGACTTACGGTTTGGATTCCTGGGGCCAAGGGGTC
GTCGTCACCGTCTCCTCA
>CM2.3 heavy chain
GANGTGCAGCTGGTGGAGTCTGGGGGCGGCTTGGCAAAGCCTGGGGGGTCCCTGAGACTC
TCCTGTGCAGCCTCTGGATTCACCTTCAGTAGCTACTACATGTACTGGGTCCGCCAGGCT
CCAGGGAAGGGGCTAGAGTGGGTCTCAGCTATTAATACTGGTGGGGGTAGCACATACTAC
GCAGACTCCGTGAAGGGCCGATTCACCATCTCCAGAGACAACTCAAAGAACACGCTCTCC
CTGCAAATGAACAGCCTGAGAGCTGAGGACACGGCCGTGTATTACTGTGCGAAAGATCGG
CAAGTATTACAATTTTTGGACTGGCTGACTTACGGTTTGGATTCCTGGGGCCAAGGGGTC
GTCGTCACCGTCTCCTCA
>CM2.2 heavy chain
GANGTGCAGCTGGTGGAGTCTGGGGGCGGCTTGGCAAAGCCTGGGGGGTCCCTGAGACTC
TCCTGTGCAGCCTCTGGATTCACCTTCAGTAACTACTACATGTACTGGGTCCGCCAGGCT
CCAGGGAAGGGGCTAGAGTGGGTCTCAGCTATTAATACTGCTGGGGGTTACACATACTAC
GCAGACTCCGTGAAGGGCCGATTCACCATCTCCAGAGACAACTCAAAGAACACGCTCTCC
CTGCAAATGAACAGCCTGAGAGCTGAGGACACGGCCGTGTATTACTGTGCGAAAGATCGG
CAAGTATTACAATTTTTGGACTGGCTGACTTACGGTTTGGATTCCTGGGGCCAAGGGGTC
GTCGTCACCGTCTCCTCA
>CM24 heavy chain
GAGGTGCAGCTGGTGGAGTCTGGGGGAGGCTTGGTCCAGCCTGGCGGGTCCCTGAGACTC
TCCTGTGCAGCCTCTGGATTCACCTTCAGTGACCACTACATGAGCTGGGTCCGCCAGGCT
CCAGGGAAGGGGCCGGAGTGGATAGGTTTCATTAGAAACCAAGCTAATGGTGGGACAGCA
GAATACGCCGCGTCTGTGAAAGGCAGATTCACCATCTCAAGAGATGATTCCAAAACCATT
GCCAGTCTCCAAATGAACAGCCTGAACACCGAAGACACGGCCGTGTATTATTGTGTTAGA
GGGGGTACCGGCGGGGGGAGGTACTGGGGCCAGGGAGTCCTGGTCACCGTCTCCTCA
>CM12.1 heavy chain
GAGGTGCAGCTGGTGGAGACTGGAGGAGGCTTGGTCCAGCCTGGGGGGTCCCTGAAACTC
TCCTGTGCAGCCTCTGGATTCACCTTCAGTAGCTATGGCATGAGCTGGGTCCGCCAGGCT
CCAGGGAAGGGGCTAGAGTGGGTCTCATCTATTAATAGTGGTGGGGGTAGCACACACTAC
GCAGACTCCGTGAAGGGCCGATTCACCATCTCCAGAGACAACTCAAAGAACACGCTCTCC
CTGCAAATGAACAGCCTGAGAGCTGAGGACACGGCCGTGTATTACTGTGCGAAATTTGGG
TGGGCGGTGCCTGGCCTTGACTATTGGGGCCAGGGAGTCCTGGTCACCGTCTCCTCA
>CM4 heavy chain
CAGGTGCAGCTGCAGGAGTCGGGCCCAGGAGTGGTGAAGCCTTCGGAGACCCTGTCCCTC
ACCTGCGCTGTCTCTGGTGGCTCCATCAGCGATAGTTACCGGTGGAGCTGGATCCGCCAG
CCCCCAGGGAAGGGACTGGCGTGGATTGGATACATCTATGGTAGTGCTACGAGCACCAAC
TACAACCCCTCCCTCAAGAGTCGAGTCACCATTTCAAAAGACACGTCCCAGAACCAGTTC
TCCTTGAAGCTGAGCTCTGTGACCGCCGCGGACACGGCCGTGTATTACTGTGCGAGCGAA
TATTACTATAGTGGTACTTATTACGGATACTACGGTTTGGATTCCTGGGGCCAAGGGGTC
GTCGTCACCGTCTCCTCA
>CM5 heavy chain
GAGGTGCAGCTGGTGGAGTCTGGGGGAGGCTTGGTCCAGCCTGGCGGGTCCCTGAGACTC
TCCTGTGCAGCCTCAGGATTCACCTTCAGTGACTACTACATGCAGTGGGTCCGCCAGGCT
CCAGGAAAGGGGCCGGAGTGGGTAGGTTTCATTAGAAACAAAGCTAATGGTGGGACAGCA
GAATACGCCGCGTCTGTGAAAGGCAGATTCACCATCTCAAGAGATGATTCCAAAAGCATT
GCCAGTCTGCAAATGAACAGCCTGAAAACCGAGGACACGGCCGTGTATTACTGTGCTAGA
GTCAGTTACGTATTACAATTTTTGGAGTGGTTAACCCTTGACTACTGGGGCCAGGGAGTC
CTGGTCACCGTCTCCTCA
>CM23 heavy chain
CAGGTGCAGCTGCAGGAGTCGGGCCCAGGAGTGGTGAAGCCTTCGGAGACCCTGTCCCTC
ACCTGCGCTGTCTCTGGTGGCTCCATCAGCGATAGTTACCGGTGGAGCTGGATCCGCCAG
CCCCCAGGGAAGGGACTGGAGTGGATTGGGTACATCTATGGTAGTAGTACGAGCACCAAC
TACAACCCCTCCCTCAAGAGTCGAGTCACCATTTCAAAAGACACGTCCAAGAACCAGTTC
TCCTTGAAGCTGAGCTCTGTGACCGCCGCGGACACGGCCGTGTATTACTGTGCGAGAGAT
GGGGGCGGCTTTACAATTTTTGGAGTGGTTATGGTATTTGACTACTGGGGCCAGGGAGTC
CTGGTCACCGTCTCCTCA
>CM11.1 heavy chain
GAGGTGCAGCTGGTGGAGTCTGGGGGAGGCTTGGTCCAGCCTGGGGGGTCCCTGAGACTC
TCCTGTGCAGCCTCTGGATTCACCTTTGGTGATTATGGCATGCACTGGGTCCGCCAGGCT
CCGGGAAAGGGGCTGGAATGGGTCTCAGCCATTAGTAGTGGTAGTGGTTACATATACTAT
GCTGACTCCGTGAAGGGCCGATTCACCATCTCCAGAGACAACGCCAAGAACTCGCTGTCA
CTGCAAATGAGCAGCCTGCGAGCCGAGGACACGGCCGTGTATTACTGTACTAGAGGCCGG
GAATACAGCGGGTACCGAGAATTTGACTTCTGGGGCCAGGGAGTCCTAGTCACCGTCTCC
TCA
>CM20 heavy chain
CAGGTGCAGCTGCAGGAGTCGGGCCCAGGACTGGTGAAGCCTTCGGAGACCCTGTCCCTC
ACCTGCACTGTCTCTGGTGGCTCCTTCAGCGGTTACTACTGGGGCTGGATCCGCCAGCCC
CCAGGGAAGGGACTGGAGTGGATTGCATATATCAGTGGTAGTAGTGGGAACACCGACTAC
AACCCGTCCCTCAAGAGTCGAGTCACCATTTCAACAGACACGTCCAAGAACCAGTTCTCC
CTGAAGCTGAGCTCTGTGACCGCCGCGGACACGGCCGTGTATTACTGTGCGAGAGTAGGA
TACAATTTTTGGACGGGTCCGGATGTCTGGGGCCGGGGAGTTCTGGTCACCGTCTCCTCA
>CM17 heavy chain
GAGGTGCAGCTGGTGGAGACTGGAGGAGGCTTGGTCCAGCCTGGGGGGTCCCTGAGACTC
TCCTGTGCAGCCTCTGGATTCACCTTCAGTAGCTATGCTATGCAGTGGGTCCGCCAGGCT
CCAGGGAAGGGGCTGGAGTGGATCTCAGCTATTAATAGTGGTGGGGGTAGCACATACTAC
GCAGACTCCGTGAAGGGCCGATTCACCATCTCCAGAGACAACTCAAAGAACACGCTCTCC
CTGCAAATGAACAGCCTGAGAGCTGAGGACACGGCCGTGTATTACTGTGCGAAAGATCTC
CTGGGGGGTAGCAGCTGGTCCAACTCATTGGATGTCTGGGGCCGGGGAGTTCTGGTCACC
GTCTCCTCA
>CM13 heavy chain
GAGGTGCAGATGGTGGAGTCTGGGGGAGGCTTGGTCCAGCCTGGGGGGTCCCTGAGACTC
TCCTGTGCAGCCTCTGGATTCACCTTTGGTGATTATGGCATGCACTGGGTCCGCCAGGCT
CCGGGAAAGGGGCTGGAGTGGGTCTCATCCATTAGTAGTGATAGTAGTTACATATACTAC
CCTGACTCCGTGAAGGGCCGATTCACCATCTCCAGAGACAACGCCAAGAACTCGCTGTCT
CTGCAAATGAGGAGCCTGAGAGCCGAGGACACGGCCGTGTATTACTGTACTAGAGATCGG
GGATCCCGGTTCGATGTCTGGGGCCCGGGAGTCCTGGTCACCGTCTCCTCA
>CM9 heavy chain
GAGGTGCAGCTGGTGGAGTCTGGGGGAGGCTTGGTCCAGCCTGGAGGGTCCCTGAGACTC
TCCTGTGCAGCCTCTGGATTCACCTTCAGTAATTACGACATGAGCTGGGTCCGCCAGGCT
CCGGGAAAGGGGCTGGAGTGGGTCTCATATGTAAGTTACACTGGTAAAACCATATACTAC
GCTGACTCCGTGAAGGGCCGATTCACCATCTCCAGAGACAACGCCAAGAAGTCGCTGTCT
CTGCAAATGAGCAGCCTGAGAGCCGAGGACACGGCCGTGTATTATTGTACTAGAGGAAAT
CCCCCAGTACAATTTTTGGAGTGGTTACCAATTGACTACTGGGGCCAGGGAGTCCTGGCC
ACCGTCTCCTCA
>CM6 heavy chain
CAGGTGCAGCTGCAGGAGTCGGGCCCACGACTGGTGAAGCCTTCGGAGACCCTGTCCCTC
ACTTGCGCTGTCTCTGGGGGCTCCATCAGCGATAGTTACTACTGGAGCTGGATCCGCCAG
TCCCCAGGGAAGGGACTGGAGTGGATTGGGTACATCTATGGTAGTGGTGGTAGCACCTAC
TACAACCCCTCCCTCAAGAGTCGAGTCACCATTTCAACAGACACATCCAAGAACCAGTTC
TCCCTGAAGCTGAGCTCTGTGACCGCCGCGGACACGGCCGTGTATTACTGTGCGAGAGAT
AATAGGCTTACAATTTTTGGACTGACATTCATTGACTACTGGGGCCAGGGAGTCCTGGTC
ACCGTCTCCTCA
>CM10 heavy chain
GANGTGCAGCTGGTGGAGTCTGGGGGAGGCTTGGTCCGGCCGGGGGGGTCCCTGAGACTC
TCCTGTGCAGCCTCTGGATTCACCTTTGGGGATTATGGCATGCACTGGGTCCGCCAGGCT
CCGGGAAAGGGGCTGGAGTGGGTCTCAACCATTAGTAGTGGTAGTGGTTACATATACTAC
GCTGACTCCGTGAAGGGCCGATTCACCATCTCCAGAGACAACGCCAAGAACTCGCTGTCT
CTGCAAATGAGCAGCCTGAGAGTCGAGGACACGGCCGTGTATTACTGTTCTAGAGGGGGG
ATACCAGCTGGTCCAAATAGATACGGTTTGGATTCCTGGGGCCAAGGGGTCGTCGTCACC
GTCTCCTCA
>CM12.2 heavy chain
GAGGTGCAGCTGGTGGAGACTGGAGGAGGCTTGGTCCAGCCTGGGGGGTCCCTGAAAGTC
TCCTGTGCAGCCTCTGGATTCACCTTCAGTACCTATGGCATGAGCTGGGTCCGCCAGGCT
CCAGGGAAGGGCCTAGAGTGGGTCTCATCTATTAGTAGTGGTGGGGTTACACACTACGCA
GACTCCGTGAAGGGCCGATTCACCATCTCCAGAGACGACTCAAAGAACACGCTCTCCCTG
CAAATGAACAGCCTGAAAACTGAGGACACGGCCGTGTATTACTGTGCGAAGTTTGGGTGG
GCGGTGCCTGGCCTTGACTATTGGGGCCAGGGAGTCCTGGTCACCGTCTCCTCA
>CM18 heavy chain
CAGGTGCAGCTGCAGGAGTCGGGCCCAGGACTGGTGAAGCCTTCGGAGACTCTGTCCCTC
ACCTGCGCTGTCTCTGGTGGCTCCTTCAGCGGTTACTACTGGGGCTGGATCCGCCAGCCC
CCAGGGAAGGGACTGGAGTGGATTGGGTATATCAGTGGTAGTAGTGGGAGCACCGACTAC
AACCCGTCCCTCAAGAGTCGAGTCACCATTTCAACAGACACGTCCAAGAACCAGTTCTCC
CTGAAGCTGAACTCTGTGACCGCCGCGGACACGGCCGTGTATTACTGTGCGAGATTGGGC
GTTAGCGGCAGCTGGTTTTGGTTCCGGTTCGATGTCTGGGGCCCGGGAGTCCTGGTCACC
GTCTCCTCA
>CM14 heavy chain
GAGGTGCAGCTGGTGGAGTCTGGGGGAGGCTTGGTCCAGCCTGGAGGGTCCCTGAGACTC
TCCTGTGCAGCCTCTGGATTCACCTTCAGTAGTTACGGCATGAGCTGGGTCCGCCAGGCT
CCGGGAAAGGGGCTGGAGTGGGTCTCATCCATTAGTAGTGCTAGTAGTTACATATACTAC
GCTGACTCCGTGAAGGGCCGATTCACCATCTCCAGAGACAACGCCAAGAACTCGCTGTCT
CTGCAAATGAACAGCCTGAGAGCCGAGGACACGGCCGTGTATTACTGTACTAGTGGGGGG
TCGGGCAGCGGCTGGTCAACCCTGAGTTTTGACTACTGGGGCCAGGGAGTCCTGGTCACC
GTCTCCTCA
>CM22 heavy chain
GAGGTGCAGCTGGTGCAGTCTGGAGCAGAGGTGAAAAGGCCCGGGGAATCTCTGAGGATC
TCCTGTAAGACTTCTGGATACAGCTTTACCGTCAGCTGGATCAGCTGGGTGCGCCAGATG
CCCGGGAAAGGCCTGGAGTGGATGGGGAGCATCTATCCTGGTGATTCTGATACCAGATAC
CACCCGTCCTTCCAAGGCCACGTCACTATCTCAGCCGACAAGTCCATCAGCACCACCTAC
CTGCAGTGGAGCAGCCTGAAGGCCTCGGACACTGCCACGTATTACTGTGCGAAAGCCCGG
TGGTCTATCTTCTACGGTTTGGATTCCTGGGGCCAAGGGGTCGTCGTCACCGTCTCCTCA
>CM19 heavy chain
CAGGTGCAGCTGCAGGAGTCGGGCCCAGGACTGGTGAAGCCTTCGGAGACCCTGTCCCTC
ACCTGCGCTGTCTCTGGGGGCTCCATCAGCGATAGTTACTACTGGAGCTGGATCCGCCAG
TCCCCAGGGAAGGGACTGGAGTGGATTGGGTACATCTATGGTAGTGGTGGTAGCACCTAC
TACAACCCCTCCCTCAAGAGTCGAGTCACCATTTCAACAGACACATCCAAGAACCAGTTC
TCCCTGAAGCTGAGCTCTGTGACCGCCGCGGACACGGCCGTGTATTACTGTGCGAGAGAT
CCCCTCTTACAATTTTTGGACTGGTTATTCATGGATGTCTGGGGCCGGGGAGTTCTGGTC
ACCGTCTCCTCA
>CM27 heavy chain
GAGGTGCAGCTGGTGGAGTCTGGGGGAGACTTGGTCCAGCCTGGCGGGTCCCTGAGACTC
TCCTGTGCAGCCTCTGGATTCACCTTCAGTGACTATGCCATGAGCTGGGTCCGCCAGGCT
TCTGGGAAAGGACTGGAGTGGGTTGGCTCTATTAGAAGCAAATATAACAATTACGCGACA
GAATACGCCGCATCGGTGAAAGGCAGATTCACCATCTCCAGAGATGATTCAAAGAACACA
CTATATCTACAAATGAACAGCCTGAAAACCGAGGACACGGCCGTGTATTACTGCACCACA
GATCACGATACTACAATTTTTGGAGTGGTTATCCCGTACTACGGTTTGGATTCCTGGGGC
CAAGGGGTCGTCGTCACCGTCTCCTCA
>CM16 heavy chain
CAGGTGCAGCTGGTGCAGTCTGGGGCTGAGGTGAAGAAGCCTGGATCCTCAGTGAAGGTC
TCCTGCAAGGCTTCCGGATACACCTTCACTGATTACTACATGCACTGGGTGCGACAGGCC
CCTGGACAAGGGCTTGAGTGGATGGGAGAAATCAACCCAAAAACTGGTGGCACAAACTAT
GCACAGAAGTTCCAGGGCAGAGTCACCATGACCAGAGACACATCGACGAGCACAGCCTAC
ATGGAGCTGAGCAGCCTGAGATCTGAGGACACGGCCGTGTATTACTGTGAGAGCTCGCGA
ATAGTGGGAACTAAATACTTCGAGTTCTGGGGCCAGGGCGCCCTGGTCACCGTCTCCTCC
>CM3 heavy chain
CAGGTGCAGCTGCAGGAGTCGGGCCCAGGAATGGTGAAGCCTTCGGAGACCCTGTCCCTC
ACCTGCGCTGTCTCTGGTGGCTCCATCAGCGATAGTTACCGGTGGACCTGGATCCGCCAG
CCCCCAGGGAAGGGACTGGAGTGGATTGGATACATCTATGGTAGTAGTACGACCACCAAC
TACAACCCCTCCCTCAAGAGTCGAGTCACCATTTCAAAAGACACGTCCAAGAACCAGTTC
TCCTTGAATCTGAGGTCTGTGACCGTCGCGGACACGGCCCTGTATTACTGTGCGAGTCCA
CCCGTATTACAATTTTTGGAGTGGACTCACTTCGAGGTCTGGGGCCAGGGCGCCCTGGTC
ACCGTCTCCTCC
>CM15 heavy chain
GANGTGCAGCTGGTGGAGTCTGGGGGAGGCTTGGTCCAGCCTGGCGGGTCCCTGAGACTC
TCCTGTGCAGCCTCTGGATTCATCTTCAGTGACTACTACATGAGTTGGGTCCGCCAGGTT
CCAGGGAAGGGGCCGGAGTGGGTAGGTTTCATTAGAAACAAAGCTAAAGGTGGGACAGCA
GAATACGCCGCGTCTGTGAAAGGCAGATTCACCATCTCAAGAGATGATTCCAAAAGCATT
GCCAGTCTACAAATGAACAGCCTGAACACCGAGGACACGGCCGTGTATTACTGTGTTTTT
GAACACTATAGTAGCAACTATAGGGTGGACTACTGGGGCCAGGGAGTCCTGGTCACCGTC
TCCTCA
>CM25 light chain
cagtctgtgctgactcagccggcctccctctcagcatctcctggagcatcagccagtctc
acatgcaccttcagcggcggcatcaatgtcgctggctaccacatactctggtaccagcag
aagccagggagtcctccccggtatcttctgaggtacaaatcagactcagataagggccag
ggctctggagtccccagccgcttctctggatccaaagatgcttcagcgaatacagggatt
ttacgcatctctgggctccagtctgaggatgaggctgactattactgtgccactgggcac
agcagcggttgggtattcggcggagggacccggctgaccgtccta
>CM1.1 light chain
cagtctgngntgactcagccgccctcagcatctggggcccccgggcagagggtcaccatc
tcctgcactgggagcagctccaacatcggggcggattattatgtatcctggtaccagcaa
ttcccaggaacggcccccaaactcctcatctatgaaaataataagcgaccctcaggggtt
tctgaccgattctctggctccaagtctggtacctcagcctccctgaccatcactgggctc
cagtctgaggatgaggctgattattactgcttagcatgggataacagcctgaatactgcc
ttattcggaggagggacccggctgaccgtccta
>CM1.2 light chain
cagtctgtgctgactcagccgccctcagcatctggggcccccgggcagagggtcaccatc
tcctgcactgggagcagctccaacatcggggcggattattatgtatcctggtaccagcaa
ttcccaggaacggcccccaaactcctcatctatgaaaataataagcgaccctcaggggtt
tctgaccgattctctggctccaagtctggtacctcagcctccctgaccatcactgggctc
cagtctgaggatgagactgattattactgcttagcatgggataacagcctgaatactgcc
ttattcggaggagggacccggctgaccntccta
>CM28 light chain
cagtctgtgctgactcagccggcctccctctcagcatctcctggagcatcagccagtctc
acatgcaccttcagcggtggcatcaatgtcgctggctaccacatattctggttccagcag
aagccagggagtcctccccggtatnttttgaggtacaaatcagactcagataagggccag
ggctccggagtccccagccgcttctcnggatccaaagatgcttcagcgaacacaggcatt
ttacgcatctgtgggctccagtgtgaggatgaggctgaatattantgtgccanagggcac
agcagcgnnngngtattcggnggagggacccggctaacc
>CM26 light chain
ctgtctgcatctgtaggagacacagtcaccatcacttgccgggcgagtcagagtattagc
agctggttagactggtatcagcagaaaccagggaaagcccctaaggtcctgatctataag
gggtccagtttggaaagtggggtcccctcaaggttcagcggcagtggatctgggacagat
ttcactctcaccatcagcagcctgcagcctgaagactttgcaacttattactgtctacag
tatagcagtagcccattcactttcggccccgggaccaaactggatatcaaa
>CM11.2 light chain
cagtctgngctgactcagccgccctcagtgtctggggcgcccgggcagagggtcaccatc
tcctgcactgggagcagctccaacattggaggttattatgtgcagtggtaccagcagctt
ccaggaacggcccccaaactcctcatctatgaaaataataagcgaccctcaggggtttct
gatcgattctctggctcccagtctggtacctcagcctccctgaccatcactagactccag
tctgaggatgaggctgattattactgccagtcctatgacagcagcctgagtgcttgggta
ttggangganggacccggcngannntcgan
>CM7 light chain
tcctatgagctgactcagccaccctcggtgtcagtgtccccaggacagacggccaggatc
acctgtgggggagacaacattggaagtgaagctgtgcactggtaccaacagaagccaccg
caggcccctgtgcaggtcatctatagtgatagagaacggccctcagggatccctgagcga
ttctctggctccaaatcagggaacaccgccaccctgaccatcagcggggtcgaggccggg
gatgaggctgactattactgtcaggtgnnggacattagtgggatcatgtctactngnnag
anggncccggcagacnnncntantcagcnnangnnt
>CM8 light chain
acccnntctccantctccctgtctttgtctccaggagaaagagccaccctctcctgcagg
gccagtcagagtgtcagcagcaacttagcctggtaccagcagaaacctgggcaagctccc
aggctcctcatctattatgcatccaacagggccactggtatcccagacaggttcagtggc
agtgggtctgggacagacttcactctcaccatcagcagcctggagcctgaagatgttgga
gtttattactgtcagcaggagagtaactggccgctcactttcggcggagggaccaaggtg
gagctcaaa
>CM21 light chain
cagtctgtgctgactcagccgccctcagtgtctggggcgcccgggcagagggtcaccata
tcctgcactgggagcagctccatcattggaggttattatgtgcagtggtaccagcagctt
ccaggaacggcccccaagctcctcatctatgaaaaaaataagcgaccctcaggggtttct
gatcgattctctggctcccagtctggtacctcagcctccctgaccatcactggactccag
tctgaggatgaggctgattattactgccagtcctatgacagcagcctgagtgctcttgta
ttcggaagtggcacc-aantgaccntcctcggtcagcctanng
>CM2.1 light chain
cagtctgnnctgactcagccaccctcagcgtctggggcccccgggcagagtgtcaccatc
tcctgttctggaagcagctccaacttcggaaggaattatgtatactggtaccagcacctc
tcaggaaaggcccccaaactcctcatctataataataatcagcgaccctcaggggtccct
gatcgattctctggctccaagtctggcacgtcagcctccctggccatcagtgggctccag
tccgaggatgaggctgattattactgctcagcatgggataacagcctgagcggcttattc
ggaggagggacccggctgaccgtccta
>CM2.3 light chain
cagtctgtgctgactcagccaccctcagcgtctggggcccccgggcagagtgtcaccatc
tcctgttctggaagcagctccaacatcggaagtaattatgtatactggtaccagcagctc
tcaggaaaggcccccaaactcctcatctataataataatcagcgaccctcaggggtccct
gatcgattctctggctccaagtctggcacgtcagcctccctggccatcagtgggctccag
tccgaggatgaggctgattattactgctcagcatgggataacagcctgagcggcttattc
ggaggagggacccggctgaccntccta
>CM2.2 light chain
cagtctgngntgactcagccaccctcagcgtctggggcccccgggcagactgtcaccatc
tcctgttctggaagcagctccaacatcggaagtaattctgtatactggtaccagcaactc
tcaggaaaggcccccaaactcctcatctataataataatcagcgaccctcaggggtccct
gatcgattctctggctccaagtctggcacgtcagcctccctggccatcagtgggctccag
tccgaggatgaggctgattattactgctcagcatgggataacagcctcagaggcttattc
ggaggagggacccggctgaccgtccta
>CM24 light chain
acccagnctccantctccctgcctgtcantnttggacagccggcctccatctcctgcagg
tctagtcaaagccttgtatacagtgatgggaaaacctacttgaattggttacagcagaag
tcaggccaacctccaaggcgcctaatttatcaggtttctaaccgggactntggggtccca
gacagattcagcggcagtgggacagggacagatttcacactgaaaatcagcagagtggag
gccgaggatgttggagtttattactgcatgcaaggtacacactggccgctcactttcggc
ggagggaccatggtggagctcaaa
>CM12.1 light chain
atnacccnnnctccagtctccctgcctgtcactcttggacagccggcctccatctcctgc
aggtctagtcaaagccttgtacacagtgatgggaaaacctacttgaattggttacagcag
aagccaggccaacctccaaggcgcctaatttatcaggtttctaaccgggactctggggtc
ccagacagattcagcggcagtggggcagggacagatttcacactgaaaatcagcagagtg
gaggctgaggatgttggagtttattactgcatgcaaggtacacactggccattcactttc
ggccccgggaccaaactggatatcaaa
>CM4 light chain
tnnnngncccnntnngnnctgactcagccaccctcggtgtcagtgtccccaggacagacg
gccaggatcacctgtgggggagacaagattggaagtaaatatgtgcactggtaccagcag
aagccaccgcaggcccctgtgttggtcatctattatgacagcgaacggccctcagggatc
cctgagcgattctctggctccaaatcagggaacaccgccaccctgaccatcagcggggtc
gaggccggggatgaggctgactattactgtcaggtgtgggatagtagtagtgatcattac
atcttcggtgctgggacccggctcaccgtccta
>CM5 light chain
cagtctgngctgactcagccaacctccctctcagcatctcctggagcatcagtcagactc
acctgcaccttgcgcagtggcatcagtgttggtggctacaatatacactggtaccagcag
aagccagggagtcctccccggtacctactgtactactactcagactcaaataaggcccag
ggctctggagtccccagccgcttctctggatccaaagatgcttcggccaatgcagggatt
ttactcatctctgggttccagtctgaggatgaggctgactattactgtacgacttggcac
aacaatgcttgggtattcggcggagggacccggctaaccgtcctantcagcnnaannnt
>CM23 light chain
atgacccnntctccantctccctgtctgcatctgtaggagacagagtcaccatcacttgc
cgggcaagtcagggcattagcagttatttaaattggtatcagcagaaaccggggaaagcc
cctaagctcctgatctattatgcaaacagtttggcaagtggggtcccatcaaggttcagc
ggcagtggatctgggacagaattcactctcaccatcagcagcctgcagcctgaagatttt
gcaacttattactgtcaacagggtaatagtaacccgtacagttttggccaggggaccaaa
gtggagatcaaag
>CM11.1 light chain
cagtctgngntgactcagccgccctcagtgtctggggcgcccgggcagagggtcaccatc
tcctgcactgggagcagctccaacattggaggttattatgtgcagtggtaccagcagctt
ccaggaacggcccccaaactcctcatctatgaaaataataagcgaccctcaggggtttct
gatcgattctctggctcccagtctgctacctcagcctccctgaccatcactggactccag
tctgaggatgaggctgattattactgccagtcctatgacagcagcctgagtgcttgggta
ttcggcggagggacccggctaacentcnnt
>CM20 light chain
cagtctgtgctgactcagcctcgctcagtgtctgggtctcctggacagtcggtcaccatc
tcctgcactggaaccagcagtgacatcggtggttataactatgtctcctggtaccaacag
catccaggcacagcccccaaactcatgatttatgaggtcagtaagcggccctcaggggtc
tctgatcgcttctctggctccaagtctggcaacacggcctccctgaccatctctgggctc
caggctgaggacgaggctgattattactgcagctcatatgcaggcagctacactttctta
ttcggaggagggacccggctgaccntcctn
>CM17 light chain
cagtctgngctgactcagccgccctcagcatctggggcccccgggcagagggtcaccatc
tcctgcactgggagcagctccaacatcggggcgggttattatgtatcctggtaccagcaa
ttcccaggaacggcccccaaactcctcatctatgaaaataataagcgaccctcaggggtt
tctgaccgattctctggctccaagtctggtacctcagcctccctgaccatcactgggctc
cagtctgaggatgaggctgattattactgcttagcatgggataacagcctgagtgctggc
ttattcggaggagggacccggctgaccgtccta
>CM13 light chain
aatcagtgnnnngacccccnnnctccantctccctgcctgtcacccttggagagccggcc
tccatctcctgcaggtctagtcagagcctcttggatagtgaggatggaaacacctatttg
gaatggtacctgcagaagccaggccagtctccacaactcttgatttatgaggtttccaac
cgggcctctggagtcccagacaggttcagtggcagtgggtcagacactgatttcacactg
aaaatcagcagagtggaggctgaggatgttggggtttattactgcatgcaagctctagac
tttcctccgacgttcggccaagggaccaaggtggaaatcaaa
>CM9 light chain
acccannntccattcnccctgtctttgtctccaggggaaagagccactctntcctgcagg
gccagtcagagtgtcagtagtttcttagcctggtaccagcagaaacctggccaggctccc
aggctcctcatctatggtgcatccagcagggccactggtatcccagacaggttcagtggc
agcgggtctgggacagacttcactctcaccattagcagcctggagcctgaagatgttgga
gtttatcactgctatcagcatagcagcgggtacactttcggcggagggaccaaggtggag
ctcaaa
>CM6 light chain
cagtctgtgctgactcagccaccctccctgtctgcatccccgggagcttcggccagactc
ccctgcaccctgagcagtgacctcagtgttggtagtaaaaccatgtactggtaccagcag
aagccagggagcgctcccaggttattcctgtactactactccgactcagacaagcagctg
ggacctggggtccccaatcgagtctctggctccaaggagacctcaagtaacacagcgttt
ttgctcatctctgggctccagcctgaggacgaggccgattattactgtcaggtgtatgac
agtagtgctggtctgctgaantnancnnnggctgcngnnnnnnnntnannncagcngnnn
a
>CM10 light chain
gtctccctgcccgtcacccctggagagccggcctccatctcctgcaggtctagtcagagc
ctcctgcatagtggtggaaaaacctatttgtattggtacctacagaagccaggccagtct
ccacagctcttgatctatgaggtttccaaccgggcctctggagtccctgacaggttcact
ggcagtgggtcaggcactgatttcaccctgagaatcagccgggtggaggctgaggatgtt
ggggtttattactgcatgcaaggtatacagcttccgtggacgttcggccaagggaccaag
gtggaaatcaaa
>CM12.2 light chain
cngtctccattcnccctgccngtcactcttggacagccggcctccatctcctgcaggtct
agtcaaagccttgtacacagtgatgggaaaacctacttgaattggttacagcagaagcca
ggccaacctccaaggcgcctaatttatcaggtttctaatcgggactctggggtcccagac
agattcagcggcagtggggcagggacagatttcacactgaaaatcagcagagtggaggct
gaggatgttggagtttattantgcatgcaaggtacacactggccattcactttnggcccc
gggaccaaactggatatcaaa
>CM18 light chain
acccagtntccnnncnccctgtctgcatcggtaggagacagagtcaccatcacttgccgg
gcaagtcagggcattagcgattatttaaattggtatcagcagaaaccaggaaaagctcct
aacctcctgatctatgctgcatccagtttgcaaagtggggtcccatcaaggttcagcggc
agtggatctgggacagatttcactctcaccatcagcagcctgcagcctgaagattttgca
gcttattactgtctacagggttatagtaccccgtacagttttggccaggggaccaaagtg
gagatcaaa
>CM14 light chain
tatgancccnnnnntccantcnccctgtctgcatctgtaggagacacagtcaccatcact
tgccgggcgagtcagagtattagcagctggttagcctggtatcagcagaaaccagggaaa
gcccctaagctcctgatctataaggcgtccagtttgcaaagtggggtcccatcaaggttc
agcggcagtggatctgggacagatttcactctcaccatcagcagcctgcagcctgaagat
ttcgcagcttattactgtctacagtatagcagtagcccattcactttcggccccgggacc
aaactggatatcaaa
>CM22 light chain
tatgaccccnannctccagtcaccctgtctgcatctgtgggagacagagtcaccatcact
tgtcgggcaagtcagggcattagcagttatttagcctggtatcagcagaaaccagggaaa
gcccctaagctcctgatctatgctgcatccactttgcaaagtggggtcccatcaaggttc
agcggcagtggatctgggacagatttcactctcaccatcagcagcctgcagcctgaagat
tttgcaacttattactgtcagcagcataatagttaccctcggacgttcggccaagggacc
aaggtggaaatcaaa
>CM19 light chain
tatgaccccnnnnntccnnnnnccctgtctgcatctgtgggagacagagtcaccatcact
tgtcgggcaagtcagggcattagcagttatttagcctggtatcagcagaaaccagggaaa
gcccctaagctcctgatctatgctgcatccactttgcaaagtggggtcccatcaaggttc
agcggcagtggatctgggacagatttcactctcaccatcagcagcctgcagcctgaagat
tttgcaacttattactgtcagcagcataatagttaccctcggacgttcggccaagggacc
aaggtggaaatcaaa
>CM27 light chain
cagtctgtgntgactcagccaccttctgcatcggcctccctgggagcctcagtcacactc
acctgcaccctgagcagcgactacagtaattatgcggtggactggcacccgcagagacca
gggaagggcccccagtttgtgatgcgagtgggctcaggtgggattgtgggatccaagggg
gatggcatccctgatcgcttctcaggctcgggctccggcctgatccggtacctgaccatc
aagaacattcaggaagaggatgagagtgactaccactgtggggcagaccacggcactggg
agcagcttcgtgttgggtattcggcggagggacccggctgaccntccta
>CM16 light chain
acccagnntccantcnccctgcccgtcacccctggagagccggcctccatctcctgcagg
tctagtcagagcctcctgcatagtaatggatacacctatttgttttggtacctgcagaag
ccaggccagtctccacagctcctgatctatttgggttccaaccgggcctctggagtccca
gacaggttcagtggcagtgggtcaggcactgatttcacactgaaaatcagccgggtggag
gctgaggatgttggggtttattactgcttgcaagatatacagcttccgctcactttcggc
ggagggaccaaggtggagctcaaa
>CM3 light chain
tatgacccnnnnnntccagtctccctgtctgcatctgtaggagacagagtcaccatcact
tgccgggcaagtcagggcattagcagttatttaaattggtatcagcagaaaccggggaaa
gcccctaacctcctgatctattatacaaacaatttggcaagtggggtcccatcaaggttc
agcggcagtggatctgggacagagttcactctcaccatcagcagcctgcagcctgaagat
tttgcgacttattactgtcaacagggtaatagttacccttacagttttggccaggggacc
aatttggagatcaaa
>CM15 light chain
cagtctgngntgactcagccgccctcagtgtctggggaccccgggcagagggtcaccatc
tcgtgcactgggagcagctccaacattggaccttattatgtatactggtaccaacaattc
ccaggaacagcccccaaactgctcatctatgacaataataagcgaccctcaggggtttct
gaccgattctctggctccaagtctggtacctcagcctccctgaccatcactgggctccag
cctggggatgaggctgattattactgctcagcatgggatagcagcctgagtgctgtgtta
ttcggaggagggacccggctgaccgtccnn

### REFERENCES

1. Smith C E, Simpson D I, Bowen E T, Zlotnik I. 1967. Fatal human disease from vervet monkeys. Lancet 2:1119-1121.
2. Siegert R, Shu H L, Slenczka W, Peters D, Müller G. 1967. [On the etiology of an unknown human infection originating from monkeys]. Dtsch Med Wochenschr 92:2341-2343. https://doi.org/10.1055/s-0028-1106144
3. Ristanović E S, Kokoškov N S, Crozier I, Kuhn J H, Gligić A S. 2020. A forgotten episode of Marburg virus disease: Belgrade, Yugoslavia, 1967. Microbiol Mol Biol Rev 84: e00095-19. https://doi.org/10.1128/MMBR.00095-19
4. Kissling R E, Robinson R Q, Murphy F A, Whitfield S G. 1968. Agent of disease contracted from green monkeys. Science 160:888-890.
5. Kissling R E, Robinson R Q, Murphy F A, Whitfield S. 1968. Green monkey agent of disease. Science 161:1364.
6. Baseler L, Chertow D S, Johnson K M, Feldmann H, Morens D M. 2017. The pathogenesis of Ebola virus disease. Annu Rev Pathol 12:387-418. Full-Length Text Journal of Virology. July 2024 Volume 98 Issue 7 10.1128/jvi.00155-2421
7. Kuhn J H, Amarasinghe G K, Basler C F, Bavari S, Bukreyev A, Chandran K, Crozier I, Dolnik O, Dye J M, Formenty P B H, Griffiths A, Hewson R, Kobinger G P, Leroy E M, Mühlberger E, Netesov [symbol] S V, Palacios G, Pályi B, Pawȩska J T, Smither S J, Takada [symbol] A, Towner J S, Wahl V, Ictv Report Consortium. 2019. ICTV virus taxonomy profile: profile: Filoviridae. J Gen Virol 100:911-912.
8. History of Marburg virus disease (MVD) outbreaks. 2021. Centers for Disease Control and Prevention.
9. History of Ebola virus disease (EVD) outbreaks. 2021. Centers for Disease Control and Prevention.
10. Hashiguchi T, Fusco M L, Bornholdt Z A, Lee J E, Flyak A I, Matsuoka R, Kohda D, Yanagi Y, Hammel M, Crowe Jr J E, Saphire E O. 2015. Structural basis for Marburg virus neutralization by a cross-reactive human antibody. Cell 160:904-912.
11. Flyak A I, Ilinykh P A, Murin C D, Garron T, Shen X, Fusco M L, Hashiguchi T, Bornholdt Z A, Slaughter J C, Sapparapu G, Klages C, Ksiazek T G, Ward A B, Saphire E O, Bukreyev A, Crowe Jr J E. 2015. Mechanism of human antibody-mediated neutralization of Marburg virus. Cell 160:893-903.
12. Marzi A, Haddock E, Kajihara M, Feldmann H, Takada A. 2018. Monoclonal antibody cocktail protects hamsters from lethal Marburg virus infection. J Infect Dis 218: S662-S665.
13. Hevey M, Negley D, Schmaljohn A. 2003. Characterization of monoclonal antibodies to Marburg virus (strain Musoke) glycoprotein and identification of two protective epitopes.
Virology 314:350-357.
14. Froude J W, Pelat T, Miethe S, Zak S E, Wec A Z, Chandran K, Brannan J M, Bakken R R, Hust M, Thullier P, Dye J M. 2017. Generation and characterization of protective antibodies to Marburg virus. MAbs 9:696-703.
15. Kajihara M, Marzi A, Nakayama E, Noda T, Kuroda M, Manzoor R, Matsuno K, Feldmann H, Yoshida R, Kawaoka Y, Takada A. 2012. Inhibition of Marburg virus budding by non-neutralizing antibodies to the envelope glycoprotein. J Virol 86:13467-13474.
16. Bozhanova N G, Sangha A K, Sevy A M, Gilchuk P, Huang K, Nargi R S, Reidy J X, Trivette A, Carnahan R H, Bukreyev A, Crowe Jr J E, Meiler J. 2020. Discovery of Marburg virus neutralizing antibodies from virus-naive human antibody repertoires using large-scale structural predictions. Proc Natl Acad Sci USA 117:31142-31148.
17. Ilinykh P A, Huang K, Santos R I, Gilchuk P, Gunn B M, Karim M M, Liang J, Fouch M E, Davidson E, Parekh D V, Kimble J B, Pietzsch C A, Meyer M, Kuzmina N A, Zeitlin L, Saphire E O, Alter G, Crowe Jr J E, Bukreyev A. 2020. Non-neutralizing antibodies from a Marburg infection survivor mediate protection by F C-Effector functions and by enhancing efficacy of other antibodies. Cell Host Microbe 27:976-991.
18. Fusco M L, Hashiguchi T, Cassan R, Biggins J E, Murin C D, Warfield K L, Li S, Holtsberg F W, Shulenin S, Vu H, Olinger G G, Kim D H, Whaley K J, Zeitlin L, Ward A B, Nykiforuk C, Aman M J, Berry J D, Saphire E O. 2015. Protective mAbs and cross-reactive mAbs raised by immunization with engineered Marburg virus Gps. PLOS Pathog 11: e1005016.
19. Aman M J, Wang Y, Kailasan S, Zhao X, Galkin A, Howell K A, Saphire E O, Li Y. 2022. Broadly neutralizing binding molecules against Marburgviruses. WO2022020327A1
20. Mire C E, Geisbert J B, Borisevich V, Fenton K A, Agans K N, Flyak A I, Deer D J, Steinkellner H, Bohorov O, Bohorova N, Goodman C, Hiatt A, Kim D H, Pauly M H, Velasco J, Whaley K J, Crowe Jr J E, Zeitlin L, Geisbert T W. 2017. Therapeutic treatment of Marburg and Ravn virus infection in nonhuman primates with a human monoclonal antibody. Sci Transl Med 9: eaai8711.
21. Kimble J B, Malherbe D C, Meyer M, Gunn B M, Karim M M, Ilinykh P A, Iampietro M, Mohamed K S, Negi S, Gilchuk P, Huang K, Wolf Y I, Braun W, Crowe J E, Alter G, Bukreyev A. 2019. Antibody-mediated protective mechanisms induced by a trivalent parainfluenza virus-vectored ebolavirus vaccine. J Virol 93: e01845-18.
22. Knuf M, Habermehl P, Zepp F, Mannhardt W, Kuttnig M, Muttonen P, Prieler A, Maurer H, Bisanz H, Tornieporth N, Descamps D, Willems P. 2006. Immunogenicity and safety of two doses of tetravalent measles-mumps-rubella-varicella vaccine in healthy children. Pediatr Infect Dis J 25:12-18.
23. Tunis M C, Deeks S L, National Advisory Committee on Immunization (NACI). 2016. Summary of the National Advisory Committee on Immunization's updated recommendations on human papillomavirus (HPV) vaccines: nine-valent human papillomavirus (HPV) of minimum intervals between doses in the HPV immunization schedule. Can Commun Dis Rep 42:149-151.
24. Swenson D L, Wang D, Luo M, Warfield K L, Woraratanadharm J, Holman D H, Dong J Y, Pratt W D. 2008. Vaccine to confer to nonhuman primates complete protection against multistrain Ebola and Marburg virus infections. Clin Vaccine Immunol 15:460-467.
25. Geisbert T W, Geisbert J B, Leung A, Daddario-DiCaprio K M, Hensley L E, Grolla A, Feldmann H. 2009. Single-injection vaccine protects nonhuman primates against infection with Marburg virus and three species of Ebola virus. J Virol 83:7296-7304.
26. Afolabi M O, Ishola D, Manno D, Keshinro B, Bockstal V, Rogers B, Owusu-Kyei K, Serry-Bangura A, Swaray I, Lowe B, et al. 2022. Safety and immunogenicity of the two-dose heterologous Ad26.ZEBOV and MVA-B N-Filo Ebola vaccine regimen in children in Sierra Leone: a randomised, double-blind, controlled trial. Lancet Infect Dis 22:110-122.
27. Warfield K L, Dye J M, Wells J B, Unfer R C, Holtsberg F W, Shulenin S, Vu H, Swenson D L, Bavari S, Aman M J. 2015. Homologous and heterologous protection of nonhuman primates by Ebola and Sudan virus-like particles. PLOS One 10: e0118881.
28. Swenson D L, Warfield K L, Negley D L, Schmaljohn A, Aman M J, Bavari S. 2005. Virus-like particles exhibit potential as a pan-filovirus vaccine for both Ebola and Marburg viral infections. Vaccine 23:3033-3042.
29. Holtsberg F W, Shulenin S, Vu H, Howell K A, Patel S J, Gunn B, Karim M, Lai J R, Frei J C, Nyakatura E K, Zeitlin L, Douglas R, Fusco M L, Froude J W, Saphire E O, Herbert A S, Wirchnianski A S, Lear-Rooney C M, Alter G, Dye J M, Glass P J, WarfieldWarfield K L, Aman M J. 2016. Pan-ebolavirus and Pan-filovirus Mouse monoclonal antibodies: protection against Ebola and Sudan viruses. J Virol 90:266-278.
30. Keck Z-Y, Enterlein S G, Howell K A, Vu H, Shulenin S, WarfieldWarfield K L, Froude J W, Araghi N, Douglas R, Biggins J, Lear-Rooney C M, Wirchnianski A S, Lau P, Wang Y, Herbert A S, Dye J M, Glass P J, Holtsberg F W, Foung S K H, Aman M J. 2016. Macaque monoclonal antibodies targeting novel conserved epitopes within filovirus glycoprotein. J Virol 90:279-291.
31. Zhao X, Howell K A, He S, Brannan J M, Wec A Z, Davidson E, Turner H L, Chiang C-I, Lei L, Fels J M, et al. 2017. Immunization-elicited broadly protective antibody reveals Ebolavirus fusion loop as a site of vulnerability. Cell 169:891-904.
32. Lehrer A T, Chuang E, Namekar M, Williams C A, Wong TAS, Lieberman M M, Granados A, Misamore J, Yalley-Ogunro J, Andersen H, Geisbert J B, Agans K N, Cross R W, Geisbert T W. 2021. Recombinant protein filovirus vaccines protect cynomolgus macaques from Ebola, Sudan, and Marburg viruses. Front Immunol 12:703986.
33. Sebastian S, Flaxman A, Cha K M, Ulaszewska M, Gilbride C, Sharpe H, Wright E, Spencer A J, Dowall S, Hewson R, Gilbert S, Lambe T. 2020. A multi-filovirus vaccine candidate: co-expression of Ebola, Sudan, and Marburg antigens in a single vector. Vaccines (Basel) 8:241.
34. Herbert A S, Kuehne A I, Barth J F, Ortiz R A, Nichols D K, Zak S E, Stonier S W, Muhammad M A, Bakken R R, Prugar L I, Olinger G G, Groebner J L, Lee J S, Pratt W D, Custer M, Kamrud K I, Smith J F, Hart M K, Dye J M. 2013. Venezuelan equine encephalitis virus replicon particle vaccine protects nonhuman primates from intramuscular and aerosol challenge with Ebolavirus. J Virol 87:4952-4964.
35. Pushko P, Parker M, Ludwig G V, Davis N L, Johnston R E, Smith J F. 1997. Replicon-helper systems from attenuated Venezuelan equine. July 2024 Volume 98 Issue 7 10.1128/jvi.00155-2422. Encephalitis virus: expression of heterologous genes in vitro and immunization against heterologous pathogens in vivo. Virology 239:389-401.
36. Öhlund P, García-Arriaza J, Zusinaite E, Szurgot I, Männik A, Kraus A, Ustav M, Merits A, Esteban M, Liljeström P, Ljungberg K. 2018. DNA-launched RNA replicon vaccines induce potent anti-Ebolavirus immune responses that can be further improved by a recombinant MVA boost. Sci Rep 8:12459.
37. Mire C E, Geisbert J B, Marzi A, Agans K N, Feldmann H, Geisbert T W. 2013. Vesicular stomatitis virus-based vaccines protect nonhuman primates against Bundibugyo ebolavirus. PLOS Negl Trop Dis 7: e2600.
38. Janus B M, van Dyk N, Zhao X, Howell K A, Soto C, Aman M J, Li Y, Fuerst T R, Ofek G. 2018. Structural basis for broad neutralization of Ebolaviruses by an antibody targeting the glycoprotein fusion loop. Nat Commun 9:3934.
39. Ishola D, Manno D, Afolabi M O, Keshinro B, Bockstal V, Rogers B, Owusu-Kyei K, Serry-Bangura A, Swaray I, Lowe B, et al. 2022. Safety and long-term immunogenicity of the two-dose heterologous Ad26.ZEBOV and MVA-B N-Filo Ebola vaccine regimen in adults in Sierra Leone: a combined open-label, non-randomised stage 1, and a randomised, double-blind, controlled stage 2 trial. Lancet Infect Dis 22:97-109.
40. Agnandji S T, Loembe M M. 2022. Ebola vaccines for mass immunization in affected regions. Lancet Infect Dis 22:8-10.
41. Pollard A J, Launay O, Lelievre J-D, Lacabaratz C, Grande S, Goldstein N, Robinson C, Gaddah A, Bockstal V, Wiedemann A, Leyssen M, Luhn K, Richert L, Bétard C, Gibani M M, Clutterbuck E A, Snape M D, Levy Y, Douoguih M, Thiebaut R, EBOVAC2 EBL2001 study group. 2021. Safety and immunogenicity of a two-dose heterologous Ad26.ZEBOV and MVA-BN-Filo Ebola vaccine regimen in adults in Europe (EBOVAC2): a randomised, observer-blind, participant-blind, placebo-controlled, phase 2 trial. Lancet Infect Dis 21:493-506.
42. Urbanowicz R A, Wang R, Schiel J E, Keck Z Y, Kerzic M C, Lau P, Rangarajan S, Garagusi K J, Tan L, Guest J D, Ball J K, Pierce B G, Mariuzza R A, Foung S K H, Fuerst T R. 2019. Antigenicity and immunogenicity of differentially glycosylated hepatitis C virus E2 envelope proteins expressed in mammalian and insect cells. J Virol 93: e01403-18. https://doi.org/10.1128/JVI.01403-18
43. Brouillette R B, Maury W. 2017. Production of filovirus glycoprotein-pseudotyped vesicular stomatitis virus for study of filovirus entry mechanisms. Methods Mol Biol 1628:53-63. https://doi.org/10.1007/978-1-4939-7116-9_4
44. Li Y, Svehla K, Louder M K, Wycuff D, Phogat S, Tang M, Migueles S A, Wu X, Phogat A, Shaw G M, Connors M, Hoxie J, Mascola J R, Wyatt R. 2009. Analysis of neutralization specificities in polyclonal sera derived from human immunodeficiency virus type 1-infected individuals. J Virol 83:1045-1059.
45. Sundling C, Zhang Z, Phad G E, Sheng Z, Wang Y, Mascola J R, Li Y, Wyatt R T, Shapiro L, Karlsson Hedestam G B. 2014. Single-cell and deep sequencing of IgG-switched macaque B cells reveal a diverse Ig repertoire following immunization. J Immunol 192:3637-3644.
46. Sundling C, Li Y, Huynh N, Poulsen C, Wilson R, O'Dell S, Feng Y, Mascola J R, Wyatt R T, Karlsson Hedestam G B. 2012. High-resolution definition of vaccine-elicited B cell responses against the HIV primary receptor binding site. Sci Transl Med 4:142ra96.
47. Cale E M, Gorman J, Radakovich N A, Crooks E T, Osawa K, Tong T, Li J, Nagarajan R, Ozorowski G, Ambrozak D R, et al. 2017. Virus-like particles identify an HIV V1V2 apex-binding neutralizing antibody that lacks a protruding loop. Immunity 46:777-791.
48. Rames M, Yu Y, Ren G. 2014. Optimized negative staining: a high-throughput protocol for examining small and asymmetric protein structure by electron microscopy. J Vis Exp 15: e51087.
49. Zivanov J, Nakane T, Forsberg B O, Kimanius D, Hagen W J, Lindahl E, Scheres S H. 2018. New tools for automated high-resolution cryo-EM structure determination in RELION-3. Elife 7: e42166.
50. Wagner T, Merino F, Stabrin M, Moriya T, Antoni C, Apelbaum A, Hagel P, Sitsel O, Raisch T, Prumbaum D, Quentin D, Roderer D, Tacke S, Siebolds B, Schubert E, Shaikh T R, Lill P, Gatsogiannis C, Raunser S. 2019. SPHIRE-crYOLO is a fast and accurate fully automated particle picker for cryo-EM. Commun Biol 2:218.
51. Smith K, Garman L, Wrammert J, Zheng N Y, Capra J D, Ahmed R, Wilson P C. 2009. Rapid generation of fully human monoclonal antibodies specific to a vaccinating antigen. Nat Protoc 4:372-384.
52. Wrammert J, Smith K, Miller J, Langley W A, Kokko K, Larsen C, Zheng N Y, Mays I, Garman L, Helms C, James J, Air G M, Capra J D, Ahmed R, Wilson P C. 2008. Rapid cloning of high-affinity human monoclonal antibodies against influenza virus. Nature 453:667-671.
53. Wu X, Yang Z Y, Li Y, Hogerkorp C M, Schief W R, Seaman M S, Zhou T, Schmidt S D, Wu L, Xu L, Longo N S, McKee K, O'Dell S, Louder M K, Wycuff D L, Feng Y, Nason M, Doria-Rose N, Connors M, Kwong P D, Roederer M, Wyatt R T, Nabel G J, Mascola J R. 2010. Rational design of envelope identifies broadly neutralizing human monoclonal antibodies to HIV-1. Science 329:856-861.
54. Urbanowicz R A, McClure C P, Brown R J P, Tsoleridis T, Persson M A A, Krey T, Irving W L, Ball J K, Tarr A W. 2015. A diverse panel of hepatitis C virus glycoproteins for use in vaccine research reveals extremes of monoclonal antibody neutralization resistance. J Virol 90:3288-3301.
55. Fusco M L, Hashiguchi T, Cassan R, Biggins J E, Murin C D, Warfield K L, Li S, Holtsberg F W, Shulenin S, Vu H, Olinger G G, Kim D H, Whaley K J, Zeitlin L, Ward A B, Nykiforuk C, Aman M J, Berry J D, Saphire E O. 2015. Correction: protective mAbs and cross-reactive mAbs raised by immunization with engineered Marburg virus GPs. PLOS Pathog 11: e1005212.
56. Gilchuk P, Murin C D, Milligan J C, Cross R W, Mire C E, Ilinykh P A, Huang K, Kuzmina N, Altman P X, Hui S, et al. 2020. Analysis of a therapeutic antibody cocktail reveals determinants for cooperative and broad ebolavirus neutralization. Immunity 52:388-403.
57. Howell K A, Brannan J M, Bryan C, McNeal A, Davidson E, Turner H L, Vu H, Shulenin S, He S, Kuehne A, Herbert A S, Qiu X, Doranz B J, Holtsberg F W, Ward A B, Dye J M, Aman M J. 2017. Cooperativity enables non-neutralizing antibodies to neutralize ebolavirus. Cell Rep 19:413-424.
58. King L B, Fusco M L, Flyak A I, Ilinykh P A, Huang K, Gunn B, Kirchdoerfer R N, Hastie K M, Sangha A K, Meiler J, Alter G, Bukreyev A, Crowe Jr J E, Saphire E O. 2018. The Marburgvirus-neutralizing human monoclonal antibody MR191 targets a conserved site to block virus receptor binding. Cell Host Microbe 23:101-109.

What is claimed:

1. A marburgvirus antibody (MARV-antibody), or fragment thereof, that binds to the polypeptide of Table III.

2. The MARV-antibody, or fragment thereof, of claim 1, comprising:
(i) heavy chain amino acid sequences of monoclonal antibodies CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28; or
(ii) a heavy chain amino acid sequences that comprises at least 50%, 60%, 70%, 80%, 85%, 90%, 95%, 96%, 97%, 97%, 99%, or above 99% identity with the heavy chain amino acid sequences of monoclonal antibodies CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28.

3. The MARV-antibody, or fragment thereof, of claim 1, comprising:
(i) the light chain amino acid sequences of the disclosed monoclonal antibodies CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28; or
(ii) a light chain amino acid sequences that comprises at least 50%, 60%, 70%, 80%, 85%, 90%, 95%, 96%, 97%, 97%, 99%, or above 99% identity with the light chain amino acid sequences of monoclonal antibodies CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28.

4. The MARV-antibody, or fragment thereof, of claim 1, comprising
(i) one or more of the heavy and/or light chain CDR regions of CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28; or
(ii) one or more heavy and/or light chain CDR regions comprising at least 50%, 60%, 70%, 80%, 85%, 90%, 95%, 96%, 97%, 97%, 99%, or above 99% amino acid identity with the CDR regions amino acid sequences of CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28.

5. The MARV-antibody, or fragment thereof, of claim 1, wherein said antibody is selected from the group consisting of a polyclonal, monoclonal, humanized, monkey-human chimeric, Fab fragment, single chain, bivalent, and multivalent antibody.

6. The MARV-antibody, or fragment thereof, of claim 1, that has been modified to increase its stability.

7. A nucleic acid encoding the MARV-antibody, or fragment thereof, of claim 1.

8. The nucleic acid of claim 7, wherein said nucleic acid encodes the (i) one or more of the heavy and/or light chain CDR regions of CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28; and or
(ii) one or more heavy and/or light chain CDR regions comprising at least 50%, 60%, 70%, 80%, 85%, 90%, 95%, 96%, 97%, 97%, 99%, or above 99% amino acid identity with the CDR regions amino acid sequences of CM1.1, CMV1.2, CM2.1, CM2.2, CM2.3, CM3, CM4, CM5, CM6, CM7, CM8, CM9, CM10, CM11.1, CM11.2, CM12.1, CM12.2, CM13, CM14, CM15, CM16, CM17, CM18, CM19, CM20, CM21, CM22, CM23, CM24, CM25, CM26, CM27 and CM28.

9. A vector comprising the nucleic acid of claim 7.

10. A cell comprising the vector of claim 9.

11. A pharmaceutical composition comprising the MARV-antibody, or fragment thereof, of claim 1.

12. A method of treating or preventing clinical signs caused by marburgvirus infection in a subject in need thereof, the method comprising administering to the subject a therapeutically effective amount of the pharmaceutical composition of claim 11.

13. A kit for diagnosing infection with Marburgvirus comprising the MARV-antibody, or fragment thereof, of claim 1.

14. A kit for treating infection with Marburgvirus comprising the MARV-antibody, or fragment thereof, of claim 1.

15. A polypeptide comprising the polypeptide sequence of Table III.

16. A polypeptide, having at least 90%-99% homology to the polypeptide of claim 15.

17. The use of the polypeptide of claim 15, or a polypeptide having at least 90%-99% homology to the polypeptide of claim 15, or fragment thereof, as
(i) a probe for marburgvirus and filovirus GP antigen specific B cell sorting and monoclonal antibody recovery from animals previously infected with a filovirus or immunized with one or more filovirus antigens;
(ii) as a reagent for recovery of serum antibodies specific for marburgvirus and filovirus GP specific antigen from animals previously infected with a filovirus or immunized with one or more filovirus antigens.

18. A vaccine composition comprising the polypeptide of claim 15, a polypeptide having at least 90%-99% homology to the polypeptide of claim 15, or fragment thereof for induction of immune responses against marburgvirus and filovirus GPs.

19. A diagnostic method for detection of marburgvirus and filovirus GP-specific antibodies wherein said polypeptide of claim 15 is used as a diagnostic reagent for detection of said marburgvirus and filovirus GP-specific antibodies.

20. A kit for detecting infection with a Marburgvirus, comprising the polypeptide of claim 15.
